(19) United States
(12) Patent Application Publication
(10) Pub. No.: US 2005/0277123 A1
(43) Pub. Date: Dec. 15, 2005

(54) Variants of NEDD4L associated with hypertension and viral budding

(76) Inventors: Robert B. Weiss, Salt Lake City, UT (US); Jean-Marc Lalouel, Salt Lake City, UT (US); James Pankow, Eagan, MN (US); Diane M. Dunn, Salt Lake City, UT (US)

Correspondence Address: TRASK BRITT, P.O. BOX 2550, SALT LAKE CITY, UT, 84110, US

(21) Appl. No.: 10/928,446

(22) Filed: Aug. 26, 2004

Related U.S. Application Data
(63) Continuation of application No. PCT/US03/06869, filed on Feb. 26, 2003.
(60) Provisional application No. 60/359,741, filed on Feb. 26, 2002.

(51) Int. Cl. C12Q 1/68; C07H 21/04; C12P 21/06; C07K 14/705

(52) U.S. Cl. 435/6; 530/350; 435/69.1; 435/320.1; 435/325; 536/23.5

(57) ABSTRACT

Disclosed are compositions and methods related to NEDD4L, a ubiquitin ligase, and hypertension as well as viral budding. A systematic search for genetic polymorphism was conducted by resequencing exon and intron boundaries in human genomic DNA. Isoforms encoding a Ca²⁺-dependent lipid binding C2 domain at the N-terminus of NEDD4L were identified. Additional isoforms lacing the Ca²⁺-dependent lipid binding C2 domain were also identified. A common polymorphism was identified, Variant 13, with either G (70%) or A (30%) as the last nucleotide of exon 1, which effects splice site finction and formation of the Ca²⁺-dependent lipid binding C2 domain. Identified isoforms are present in both kidney and adrenal samples.

### CROSS REFERENCE TO RELATED APPLICATIONS

This application is a continuation of International Application Number PCT/US03/06869, filed Feb. 26, 2003, published in English Nov. 13, 2003, as International Publication Number WO 03/093452 A2, which claims the benefit under 35 U.S.C. § 119(e) of U.S. Provisional Application No. 06/359,741, filed Feb. 6,2002, the contents of both of which are incorporated by reference.

### GOVERNMENT LICENSE RIGHTS

The research supporting this invention was partially funded by National Institute of Health, grant number NIH/NHLBI 5U01-HL5449607. The United States Government may have some right in this invention.

### INCORPORATION-BY-REFERENCE OF MATERIAL SUBMITTED ON A COMPACT DISC

The Sequence Listing is provided on compact disc, under 37 C.F.R. §§ 1.821(c) and 1.823(a)(2), rather than on paper.

Pursuant to 37 C.F.R. § 1.52(e)(5), the Sequence Listing has been submitted via CD-R, and is hereby incorporated by reference in its entirety. The CD-R is labeled and contains only one 16 Mb file (274576PC.APP) recorded on Feb. 26, 2003.

### TECHNICAL FIELD

The invention relates generally to biotechnology, and more specifically to genetic traits associated with variants of NEDD4L which effect hypertension and viral budding.

### BACKGROUND

Genetic determinants of essential hypertension have proven elusive, as no single common variant is likely to exert a major effect on blood pressure (Corvol et al. 1999; Luft 2000). In rare Mendelian syndromes of hypertension, by contrast, gain or loss of function mutations account for the physiology and the molecular basis of disorders. Such remarkable advances have pinpointed critical elements of regulatory pathways involved in sodium homeostasis and blood pressure control (Lifton et al. 2001). Thus in Liddle's syndrome (Liddle et al. 1963), mutations affecting PY domains of the epithelial sodium channel (ENaC) lead to increased sodium reabsorption as a result of increased ENaC activity (Shimkets et al. 1994; Schild et al. 1995). ENaC mediates sodium reabsorption in the cortical collecting tubules of the kidney, accounting for the fine regulation of sodium balance. The cell surface expression of ENaC is regulated by hormones such as aldosterone and vasopressin and by intracellular signaling, including ubiquitination and phosphorylation (Debonneville et al. 2001; Snyder et al. 2002)

### SUMMARY OF THE INVENTION

Disclosed herein are common genetic traits associated with variants of a protein called NEDD4L which effect hypertension through, for example, Na transport through the ENaC, and viral budding.

Described is an isolated nucleic acid having a sequence useful in the diagnosis of hypertension or enveloped viral infection. Particularly, the use of one or more variants selected from Table 3 or FIG. 5.

In one embodiment, the invention relates to an isolated nucleic acid comprising a nucleic acid encoding a NEDD4L gene product having a Ca²+-dependent lipid binding (C2) domain or a fragment thereof.

Another embodiment of the invention relates to a vector or an expression vector having a nucleic acid of the invention.

Another embodiment of the invention relates to a host cell having an isolated nucleic acid of the invention. The host cell may be E. Coli, Bacillus sp., Streptomyces sp., yeast, fungi, insect cells, plant cells, mammalian cells, or the like.

Another embodiment of the invention relates to NEDD4L polypeptides having a having a Ca²⁺-dependent lipid binding (C2) domain or a fragment thereof.

Another embodiment of the invention relates to an antibody capable of recognizing a fragment or epitope derived from NEDD4L and/or the Ca²⁺-dependent lipid binding C2 domain of NEDD4L.

Another embodiment of the invention relates to a composition for identifying sequence information about a NEDD4L gene. This embodiment encompasses primers capable of providing sequence information, particularly, about position variant 13 and probes, for example, DNA and RNA probes capable of providing sequence information about NEDD4L.

Another embodiment of the invention relates to a composition for identifying sequence information about a NEDD4L gene, wherein the primer provides direct sequence information about variant 13.

Another embodiment of the invention relates to a composition for identifying sequence information about a NEDD4L gene, wherein the primer provides sequence information about one or more variants listed in Table 3 and/or a microsatellite polymorphism, for example, a GT microsatellite.

Another embodiment of the invention relates to identifying sequence information about a NEDD4L gene using genetic linkage to a single nucleotide polymorphism, a restriction fragment polymorphism, a dinucleotide polymorphism, a trinucleotide polymorphism, a deletion or an insertion.

Another embodiment of the invention relates to a method for diagnosing, prognosing and/or treating hypertension or viral infection. Hypertension is linked to NEDD4L and the invention discloses methods for treating hypertension based on information regarding NEDD4L.

Additional advantages of the invention will be set forth in part in the description, which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

The accompanying drawings, which are incorporated in and constitute a part of this specification, illustrate several embodiments of the invention and together with the description, serve to explain the principles of the invention.

FIG. 1 shows the Exon 1-exon 2 splice junctions and predicted translation (A) found from RT-PCR products of kidney and adrenal total RNA using exon 1 and exon 3 primers (SEQ ID NOS:135, 134, 137, 136, 132-133, 139, 138, respectively, in order of appearance). The number of independent sequence-verified clones (B) containing either variant 13 G or A in splice products 1 or 2 is shown for kidney and adrenal RNA preparations. The p-value from Fischer's exact test of whether variant 13 affects splice site selection is also shown.

FIG. 2 shows 5′ RACE analysis of NEDD4L in kidney and adrenal gland. The exonic start location of the most 5′ end for each transcript is shown, as well as the total number of clones and the number of unique 5′ ends found for each transcript. The exonic coordinates locate the 5′ end in the human genome draft assembly hg8 06 Aug. 2001 freeze (www.genome.ucsc.edu, University of California, Santa Cruz) by addition of 65,000,000 bases.

FIG. 3 shows the Exon-intron structure of the 5′ end of NEDD4L on human chromosome 18q. The exons (FIG. 3A) were defined by searching GenBank Human EST entries and nr databases with NEDD4L exon 3 as the query (CAGTGATCCGTATGTGAAACTTTCATTGTACGTAGCGGATGAGAATAGA GAACTTGCTTTGGTCCAGACAAAAACAATTAAAAAG) (SEQ ID NO: 131). ESTs and cDNAs that have exons spliced 5′ to exon 3 are shown. AF21730 (SEQ ID NO: 180) sequence 5′ of exon 2 is not represented in the current draft sequence of the human genome. The sequences were compared to human genome draft assembly hg8 06 Aug. 2001 freeze to define exon-intron boundaries; all exons conformed to consensus splice sites (5′-AG, 3′-GT). The exonic location and conservation of the C2 domain (FIG. 3B) is shown for NEDD4L transcripts. The alignment of human NEDD4L (SEQ ID NO:141) is shown versus the SMART C2 (SM0239) (SEQ ID NO:140) consensus sequence. The alignments of predicted C2 domains of mouse and Fugu NEDD4L orthologs, and the human NEDD4 paralog, are also shown (SEQ ID NOS:142-151, respectively, in order of appearance).

FIG. 4 shows the results of quantitative PCR analysis of exon 1-2-3 isoform I versus exon 2a-exon3 isoform III in human kidney (hK), adrenal gland (hA), and liver (hL). Standard curves from a two-fold dilution series of cloned PCR target for both NEDD4L isoforms and human GAPDH were used to calculate the relative ratios of NEDD4L/GAPDH. The NEDD4L/GAPDH ratios are calculated based on predicted molar amounts of target mRNA. The probability that the expression levels are significantly different is shown with p values calculated with a Student's t-test.

FIG. 5 shows linkage disequilibrium between a GT microsatellite polymorphism located approximately 1.2 Kb 3′ of variant 13. 5A) shows the frequency of linkage between alleles of the microsatellite, alleles being represented by an arbitrary repeat value of between 2 and 17, and either G (solid line) or A (dashed line) of variant 13 in Caucasians. 5B) shows the frequency of linkage between alleles of the microsatellite and either G (solid line) or A (dashed line) of variant 13 in African-Americans (Blacks).

FIG. 6 shows the linkage between alleles of the microsatellite polymorphism and variant 13-G (solid lines) or variant 13-A (dashed lines). FIG. 6 is expressed as the percentage of variant 13-A or 13-G linked to each allele of the microsatellite polymorphism. Thus, correcting for the allele frequency difference between 13-A and 13-G. 5A) is the linkage disequilibrium present in Caucasians, 5B) is the linkage disequilibrium present in African-Americans (Blacks).

### DETAILED DESCRIPTION OF THE INVENTION

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific synthetic methods, specific recombinant biotechnology methods unless otherwise specified, or to particular reagents unless otherwise specified, as such may, of course, vary.

As used in the specification and the appended claims, the singular forms include the plural unless the context clearly dictates otherwise. Thus, for example, reference to “a pharmaceutical carrier” includes mixtures of two or more such carriers, and the like.

In this specification and in the claims which follow, reference will be made to a number of terms which shall be defined to have the following meanings:

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example, through hybridization. The hybridization of nucleic acids is well understood in the art and discussed herein. Typically a probe can be made from any combination of nucleotides or nucleotide derivatives or analogs available in the art.

Throughout this application, various publications are referenced. The disclosures of these publications in their entireties are hereby incorporated by reference into this application. The references disclosed are also individually and specifically incorporated by reference herein for the material contained in them that is discussed in the sentence in which the reference is relied upon.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular NEDD4L is disclosed and discussed and a number of modifications that can be made to a number of molecules including the NEDD4L are discussed, specifically contemplated is each and every combination and permutation of NEDD4L and the modifications that are possible unless specifically indicated to the contrary. Likewise, any subset or combination of these is also disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

NEDD4L

The appropriate names for the genes are NEDD4 and NEDD4L. These genes have also been referred to as Nedd4-1 and Nedd4-2, respectively. For example, there are two rather similar Nedd4 genes in humans and mouse. They are commonly referred to as Nedd4-1 and Nedd4-2. The HUGO Nomenclature substitutes NEDD4 for Nedd4-1 and NEDD4L (Nedd4-like) for Nedd4-2. NEDD4 is located on chromosome 15. NEDD4L is located in chromosome 18q, the area for which linkage has been reported to phenotypes affecting blood pressure.

As disclosed herein, NEDD4L leads to the formation of multiple isoforms, which encodes proteins that share a subset of their functional domains. In addition to the shorter isoform investigated thus far in human and mouse (isoform III), disclosed herein is the existence in humans of additional isoforms (for example, isoforms I and II) that includes an additional domain of critical significance for its function. Specifically, the isoforms disclosed encode a protein that includes a C2-domain, a calcium-dependent binding domain that, by promoting association with specific intracellular proteins or lipids, leads to targeting of the protein to the cell membrane. In particular, this domain leads to specific interactions with components of membrane microdomains referred to as “lipid rafts.”

A consequence of this cellular targeting is that NEDD4L containing the C2 domain, isoforms I & II, will be sorted to regions of the apical membrane where it will come to interact with the epithelial sodium channel (ENaC), affecting its residence time at the cell surface. This in turn exerts a direct effect on sodium reabsorption in distal nephron, with attendant effect on plasma volume and blood pressure regulation.

Disclosed herein are isoforms of NEDD4L where the gene encodes a C2 domain at the N-terminus of the gene product. As disclosed herein, NEDD4L includes isoforms with and without the C2 domain. The presence of a C2 domain has important implications for the diagnosis and treatment of hypertension and viral infection.

Budding of enveloped viruses is affected by the ubiquination state of viral proteins, such as viral membrane proteins. The Nedd4 family of ubiquitin-protein ligases (E3s), AIP4, WWP2/AIP2, and Nedd4, have been shown to specifically bind to two PY motifs present within the amino-terminal domain of the latent membrane protein 2A (LMP2A) of Epstein-Barr virus (EBV). PY motifs interact with WW domains through a consensus sequence of xPPxY. Importantly, the ubiquination pathway has been linked to retrovirus budding (Kikonyogo et al. (2001) Proc. Natl. Acad. Sci. USA 98(20):11199-204). The C2 domain of NEDD4L targets the gene product to the plasma membrane and lipid rafts. C2-NEDD4L present in, for example, lipid rafts will facilitate ubiquination of viral proteins and may increase viral production. Therefore, NEDD4L lacking a C2 domain is believed to provide protection from enveloped virus infection.

Several groups have established linkage between the region of chromosome 19 containing NEDD4L and hypertension. Sodium regulation has also been established as playing a critical role in hypertension. Sodium regulation will be effected by the residence time of ENaC at the cell surface. ENaC contains PY domains which interact with the WW domains found in NEDD4L. The ENaC-PY:WW-NEDD4L interaction is also regulated by, for example, aldosterone, which affects the activity of kinases, such as, serum glucocorticoid kinase (SGK). NEDD4L is a substrate for SGK dependent phosphorylation, which allows for increased interaction between ENaC and NEDD4L. As a consequence increased phosphorylation allows for increased ubiquination of ENaC. For example, mutations in the PY domain of ENaC are associated with Liddle's Syndrome. NEDD4L with a C2 domain is preferencially localized to the cell membrane where ENaC functions. Thus, localization of NEDD4L to the cellular membrane can decrease the residence time of ENaC and alter sodium retention.

Hypertension may be divided into two categories. The first is volume expanded, which may result from sodium retention. The second is volume constricted, which may result from vaso constriction. Typically, hypertension is treated with drugs that effect one of the two categories, however, selection of a treating drug is typically a matter of trial and error or the use of a combination of the two categories of drugs.

The invention disclosed herein allows a treating physician to make an informed choice of hypertensive treatment options. The absence of a C2 domain may lead to increased cation retention or a volume expanded condition. Such a condition may be more effectively treated with volume depleting drugs, such as diuretics and calcium channel blockers.

Treatment of hypertension in a patient suffering from a volume expanded condition with volume depleting drugs is expected to return the patient to a neutral volume or to a volume constriction condition in a patient, for example, by over correcting the volume expanded condition. Treatment of volume constricted patients with volume depleting drugs may exacerbate the volume constriction.

Hypertensive patients were screened for position variations, including position variation 13. Because the patients studied were being treated with one or more drugs they were sorted by medication type. Volume depleting drugs constitute medication group 1 and vaso dilators constitute medication group 2. As shown in Table XX, patients in medication group 1, having a G at variant 13 display a significant orthostatic hypotension response.

One common mutation disclosed herein, referred to as variant 13A, leads to the formation of an aberrant transcript that can no longer generate isoforms encoding a C2 domain. Consequently, this will adversely affect down regulation of ENaC, leading to sodium retention, plasma volume expansion and increased arterial pressure.

Variant 13A and other mutations in NEDD4L, by their effects on the number of sodium channel units present at the cell surface, have relevance in any human disease involving or affecting cation transport through ENaC, or in the susceptibility or the response to pharmacologic agents that affect transport mediated by the channel.

Also disclosed is experimental evidence that C2-containing NEDD4 homologs in humans or yeast are targeted in lipid rafts, where they interact with viral proteins involved in assembly and viral budding. Any alteration of this pathway will affect budding of enveloped viruses.

Therefore, in addition to the role of NEDD4L in blood pressure regulation, mutations in the gene, including variant 13A, are associated with effects on the transport of NEDD4L to lipid rafts, and therefore its participation in processes leading to viral budding. Therefore, mutations in NEDD4L can be associated with individual differences in susceptibility to viral infections.

Late viral bud maturation of enveloped viruses occurs in lipid rafts and viruses with late domain PY motifs are believed to require NEDD4L for efficient budding. Since the NEDD4L C2 domain is required for lipid raft targeting, then variant 13G can confer increased viral susceptibility.

The presence of variant 13G results in the targeting of C2-NEDD4L proteins to the cellular membrane and lipid rafts. NEDD4L present in lipid rafts is positioned to facilitate ubiquination of viral membrane proteins, which has been shown to effect viral budding. Therefore, inhibition of the NEDD4L C2 domain activity is an antiviral target.

The members of the Nedd4/Rsp5 protein family have a unique modular structure consisting of an N-terminal Ca²⁺/lipid-binding (C2) domain, multiple WW protein-protein interaction domains and an E3 ubiquitin ligase HECT domain. The WW domains of the NEDD4 family of ubiquitin ligases specifically interact with the PY domains in ENaC subunits leading to monoubiquitination of ENaC and down-regulation via endocytosis from the plasmid membrane. The vesicles carrying ENaC enter the vacuolar protein sorting pathway leading to recycling to the plasma membrane or targeting to lysosome for degradation (Lemmon and Traub 2000). There are two Nedd4 paralogs in human: NEDD4 (chromosome 15q, GenBank LocusID 4734) and NEDD4L (chromosome 18q, GenBank LocusID 23327). Although NEDD4 orthologs were originally identified as ENaC binding partners using yeast two-hybrid experiments (Staub et al. 1996), it has recently been established that NEDD4L orthologs (also termed human, mouse Nedd4-2 proteins) are more potent regulators of ENaC (Kamynina et al. 2001 a; Kamynina et al. 2001b; Harvey et al. 2001).

Genetic linkage in a region of human chromosome 18q has been suggested for essential hypertension (Atwood et al. 2001) and postural change in systolic blood pressure (Pankow et al. 2000), and established for orthostatic hypotensive disorder (DeStefano et al. 1998). Significant linkage has also been reported for essential hypertension (Kristjansson et al. 2002) and orthostatic hypotensive disorder (DeStefano et al. 1998) in the vicinity of the peaks of these other reports. This region includes the ubiquitin ligase NEDD4L (GenBank Locus ID 23327). The NEDD4L gene disclosed herein was surveyed for common polymorphisms by resequencing NEDD4L exons. Among multiple polymorphisms identified in the NEDD4L gene, a common variant (population frequency of 30% in Caucasians) at the exon 1 splice junction disrupts the expression of a transcript capable of producing a NEDD4L protein containing a C2 domain. Characterization of the 5′ end of the NEDD4L transcript also reveals multiple transcript isoforms that contain or lack this C2 domain.

Taken together, linkage evidence implicating chromosome 18q, the significance of NEDD4L in ENaC regulation, association with medicament treatment and orthostatic hypotension in treated hypertensive patients and the putative loss of function resulting from this common splice junction variant indicate that it contributes to essential hypertension and can play a role in viral budding.

Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid or amino acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman, Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by, for example, the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids.) A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent of the available limiting nucleic acid is bound to the non-limiting nucleic acid. Typically, the non-limiting primer is in, for example, 10 or 100 or 1000 fold excess. This type of assay can be performed under conditions where both the limiting and non-limiting primer are, for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent of the available primer is enzymatically manipulated under conditions which promote the enzymatic manipulation, for example, if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent of the primer molecules are extended. Preferred conditions also include those suggested by the manufacturer or indicated in the art as being appropriate for the enzyme performing the manipulation.

Alternatively, selective hybridization may be defined by a functional determinant. For example, primers used in a polymerase chain reaction (PCR), are said to bind specifically when amplification of the target sequence results in unique bands of the expected length.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example, if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

Nucleic Acids

There are a variety of molecules disclosed herein that are nucleic acid based, including, for example, the nucleic acids that encode NEDD4L, homologs, orthologs, or fragments of the same, gene products as well as various functional nucleic acids. The disclosed nucleic acids are made up of, for example, nucleotides, nucleotide analogs, or nucleotide substitutes. Non-limiting examples of these and other molecules are discussed herein. It is understood that, for example, when a vector is expressed in a cell, that the expressed mRNA will typically be made up of A, C, G, and U. Likewise, it is understood that if, for example, an antisense molecule is introduced into a cell or cell environment through, for example, exogenous delivery, it is advantageous that the antisense molecule be made up of nucleotide analogs that reduce the degradation of the antisense molecule in the cellular environment.

Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. A non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include, for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance, for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989,86, 6553-6556.)

Sequences

There are a variety of sequences related to the NEDD4L, homologs and orthologs, or fragments of the same, for example, having Genbank Accession Numbers discussed herein. These sequences and others are herein incorporated by reference to their Genbank Accession numbers in their entireties as well as for individual subsequences contained therein.

The particular sequences set forth herein and having the Genbank accession numbers are used herein, for example, to exemplify the disclosed compositions and methods. It is understood that the description related to this sequence is applicable to any sequence related to NEDD4L, homologs and orthologs, or fragments of the same, unless specifically indicated otherwise. Sequence discrepancies and differences can be adjusted to, and the compositions and methods relating to a particular sequence can be applied to other related sequences (i.e., sequences of NEDD4L homologs and orthologs, or fragments of the same,). Primers and/or probes can be designed for any NEDD4L, NEDD4 or ortholog sequence given the information disclosed herein.

Variant 13A Region of NEDD4L

The variant 1 3A of NEDD4L occurs at the splice junction of Exon 1. This mutation leads to an NEDD4L isoform that lacks a C2 domain. This mutation was found to be present in 30% of the caucasian and African-american population. It is understood that this variant can be assayed in any population or subpopulation of individuals given the information and direction contained herein indicating that this variant is related to hypertension. Assaying for the presence of this variant or other variants using primers or probes as described herein is desirable for determining an individual's predisposition for acquiring or being susceptible to hypertension and/or viral infection. NEDD4L has been sequenced in a large number of individuals. NEDD4L, Variant 13A, is associated with orthostatic hypotension of patients receiving volume deleting hypertension drugs.

Primers and Probes

Disclosed are compositions including primers and probes, which are capable of interacting with the NEDD4L gene, homologs and orthologs, or fragments of the same, as disclosed herein. It is understood that if the specification identifies NEDD4L gene, homologs and orthologs, or fragments of the same, that this could also refer to related nucleic acids, such as the mRNA or cDNA unless specifically indicated to the contrary or by what one of skill in the art understands. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where, for example, the nucleotides or oligonucleotides used to extend the primer are modified such that they will chemically react to extend the primer in a sequence specific manner. Typically the disclosed primers hybridize with a region or regions of the NEDD4L gene, homologs and orthologs, or fragments of the same, or they hybridize with a region or regions of the complement of the NEDD4L gene, homologs and orthologs, or fragments of the same.

The size of the primers or probes for interaction with the NEDD4L gene, homologs and orthologs, or fragments of the same, in certain embodiments can be any size that supports the desired enzymatic manipulation of the primer, such as DNA amplification or the simple hybridization of the probe or primer. A typical NEDD4L gene, NEDD4 gene or homologs and orthologs, or fragments of the same, primer or probe would be at least 6-15, 15-20, 20-25, 25-30, 30-35, 35-40, 40-45, 45-55, 55-65, 75-100, 100-200, 200-300, 300-400, 400-500, 500-600, 600-700, 700-800, 800-900, 900-1000, 1000-2000, 2000-3000 or 3000-4000 nucleotides long.

In other embodiments an NEDD4L gene, NEDD4 gene orhomologs and orthologs, or fragments of the same, primer or probe can be less than or equal to 6-15, 15-20, 20-25, 25-30, 30-35, 35-40, 40-45, 45-55, 55-65, 75-100, 100-200, 200-300, 300-400, 400-500, 500-600, 600-700, 700-800, 800-900, 900-1000, 1000-2000, 2000-3000 or 3000-4000 nucleotides long.

In certain embodiments the primers and probes are designed such that they are primers whose nearest point of interaction with the NEDD4L gene, or homologs and orthologs, or fragments of the same, is within 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 300, 400, 500, or 1000 nucleotides of the position of the variant 13 A within the NEDD4L gene, NEDD4 gene or homologs and orthologs, or fragments of the same.

For example, for a particular NEDD4L gene or homolog (identified in the chromosome 18 draft assembly hg8 from the August 6, 2001 freeze, with coordinates indexed to base 65,000,000), certain embodiments of the primer or probe would be designed such that they are primers or probes whose nearest point of interaction to the position of variant 13, or another variant listed in Table 3, as discussed herein, with this particular NEDD4L gene or homolog, would occur at about 1 bp to 1 kb from variant 13, or another variant listed in Table 3. The primer or probe may interact with either strand of a duplex DNA in either direction, 5′ or 3′, form variant 13, or another variant listed in Table 3. It is understood that there is a similar position in each homologue and ortholog of NEDD4L.

It is understood that the primer or probe can interact with the NEDD4L gene, NEDD4 gene, or homologs and orthologs, or fragments of the same, at any point such that sequence data regarding variant 13 can be obtained for any individual or DNA sample. For example, numerous variants are disclosed herein, which show genetic linkage to variant 13. A primer or probe that establishes the presence or absence of another variant is understood to provide sequence data regarding variant 13.

The primers for the NEDD4L gene, homologs and orthologs, or fragments of the same, typically will be used to produce an amplified DNA product that contains sequence information regarding the variant 13 region of the NEDD4L gene, homologs and orthologs, or fragments of the same, or such that any sequence information is obtained regarding any of the specific variants discussed in Table 3. In general, typically the size of the product will be such that the size can be accurately determined to within 3, or 2 or 1 nucleotides.

In certain embodiments the product of the primer or probe is at least 20-25, 25-30, 30-35, 35-40, 40-45, 45-55, 55-65, 75-100, 100-200, 200-300, 300-400, 400-500, 500-600, 600-700, 700-800, 800-900, 900-1000, 1000-2000, 2000-3000 or 3000-4000 nucleotides long. In other embodiments the product is less than or equal to 20-25, 25-30, 30-35, 35-40, 40-45, 45-55, 55-65, 75-100, 100-200, 200-300, 300-400, 400-500, 500-600, 600-700, 700-800, 800-900, 900-1000, 1000-2000, 2000-3000 or 3000-4000 nucleotides long.

Functional Nucleic Acids

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of NEDD4L gene, homologs and orthologs, or fragments of the same, or the genomic DNA of NEDD4L gene, homologs and orthologs, or fragments of the same, or they can interact with the polypeptide encoded by the NEDD4L gene, homologs and orthologs, or fragments of the same. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place. As discussed herein functional nucleic acids that can distinguish NEDD4L gene products, or homologs and orthologs, or fragments of the same, gene products having a C2 domain related to the splicing at Exon 1 are disclosed. These functional nucleic acids can be used in a variety of ways, including as competitive inhibitors of ENaC protein interactions.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d}) less than 10⁻⁶, preferable less than 10⁻⁸, more preferably less than 10⁻¹⁰, more preferably less than 10⁻¹². A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. Nos. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with k_{d} from the target molecule of less than 10⁻¹² M. Aptamers can bind the target molecule with a very high degree of specificity, for example, with a k_{d} less than 10⁻⁶, preferable less than 10⁻⁸, more preferably less than 10⁻¹⁰, more preferably less than 10⁻¹². For example, aptamers have been isolated that have greater than a 10,000 fold difference in binding affinities between the target molecule and another molecule that differ at only a single position on the molecule (U.S. Pat. No. 5,543,293). It is preferred that the aptamer have a k_{d} with the target molecule at least 10 to 10,000 fold lower than the k_{d} with a background binding molecule. It is preferred when doing the comparison for a polypeptide, for example, that the background molecule be a different polypeptide. For example, when determining the specificity of NEDD4L gene product, or homologs and orthologs, or fragments of the same, gene product aptamers, for example, aptamers specific for the C2 domain of NEDD4L, the background protein could be serum albumin. Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, International Publication WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity, for example, with a K_{d} less than 10⁻¹², 10⁻¹⁰, 10⁻⁸ or 10⁻⁶. Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (International Publication WO 92/03566 by Yale, and Forster and Altman, Science 238:407-409 (1990)).

Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J 14:159-168 (1995), and Carrara et al., Proc. Natl. Acad. Sci. USA 92:2627-2631 (1995).) Representative examples of how to make and use EGS molecules to facilitate cleavage of a variety of different target molecules be found in the following non-limiting list of U.S. Pat. Nos. 5,168,053, 5,624,824, 5,683,873, 5,728,521, 5,869,248, and 5,877,162.

Peptides

Protein Variants

As discussed herein there are numerous variants of the NEDD4L gene product, NEDD4 gene product, or homologs and orthologs, or fragments of the same, gene product that are known and herein contemplated. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example, M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e., a deletion of 2 residues or insertion of 2 residues. Substitutions, deletions, insertions or any combination thereof may be combined to arrive at a final construct. Substitutional variants are those in which at least one residue has been removed and a different residue inserted in its place. Such substitutions generally are made in accordance with the following Table 1 and are referred to as conservative substitutions.

TABLE 1
Amino Acid Substitutions

| | Exemplary Conservative |
|---|---|
| Original | Substitutions, others |
| Residue | are known in the art. |
| Ala | ser |
| Arg | lys, gln |
| Asn | gln; his |
| Asp | glu |
| Cys | ser |
| Gln | asn, lys |
| Glu | asp |
| Gly | ala |
| His | asn; gln |
| Ile | leu; val |
| Leu | ile; val |
| Lys | arg; gln; |
| Met | Leu; ile |
| Phe | met; leu; tyr |
| Ser | thr |
| Thr | ser |
| Trp | tyr |
| Tyr | trp; phe |
| Val | lile; leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example, as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; Phe; and Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished, for example, by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

As discussed herein, a functional fragment is part of a molecule which retains a specified function. For example, the C2 domain of the protein may be expressed as a fragment or as a part of a fusion protein, where the C2 domain retains the function of targeting the molecule to the appropriate cellular location. For example, the C2 domain may be fused to one or more other domains, whereby the one or more other domains can be targeted to the plasma membrane and/or lipid rafts. In the context of nucleic acid used to screen for the presence of variant 13 or another variant, the nucleic acid need not code for the NEDD4L gene product and must only be of sufficient size to uniquely identify the target sequence.

Antibodies

Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with NEDD4L gene, NEDD4 gene, or homologs and orthologs, or fragments of the same, or gene products such that, for example, the C2 domain is specifically recognized, as disclosed herein. The antibodies can be tested for their desired activity using the in vitro assays described herein, or by analogous methods, after which their in vivo therapeutic and/or prophylactic activities are tested according to known clinical testing methods. Also disclosed are functional equivalents of antibodies.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

The disclosed monoclonal antibodies can be made using any procedure which produces monoclonal antibodies. For example, monoclonal antibodies of the invention can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent.

The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas III et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

However, an antibody, human, humanized or non-human, can be used to screen biological samples for the presence of the antigen. For example, a monoclonal antibody can be generated which specifically recognizes the C2 domain of NEDD4L and used to screen a sample, for example, a human tissue sample, for the presence of NEDD4L having a C2 domain. The sample may be screened using methods known in the art, for example, an enzyme-linked immunosorbent (ELISA) assay, Western blots, affinity chromatography, or the like.

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods of the invention serves to lessen the chance that an antibody administered to a human will evoke an undesirable immune response.

Human Antibodies

The human antibodies of the invention can be prepared using any technique. Examples of techniques for human monoclonal antibody production include those described by Cole et al. (Monoclonal Antibodies and Cancer Therapy, Alan R. Liss, p. 77, 1985) and by Boerner et al. (J. Immunol., 147(1):86-95, 1991). Human antibodies of the invention (and fragments thereof) can also be produced using phage display libraries (Hoogenboom et al., J. Mol. Biol., 227:381, 1991; Marks et al., J. Mol. Biol., 222:581, 1991).

The human antibodies of the invention can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge.

Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fv, Fab, Fab′, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

Administration of Antibodies

Administration of the antibodies can be done as disclosed herein. Nucleic acid approaches for antibody delivery also exist. For example, antibodies that bind and/or neutralize NEDD4L gene product, NEDD4 gene product, or homologs and orthologs, or fragments of the same, can be administered either alone or with one or more adjuvants. Gene product antibodies and antibody fragments of the invention can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

Delivery of the Compositions to Cells

Nucleic Acid Delivery

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247,1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

In the methods described herein, which include the administration and uptake of exogenous DNA into the cells of a subject (i.e., gene transduction or transfection), the nucleic acids of the present invention can be in the form of naked DNA or RNA, or the nucleic acids can be in a vector for delivering the nucleic acids to the cells, whereby the encoding DNA or DNA or fragment is under the transcriptional regulation of a promoter or regulatory sequence, as would be well understood by one of ordinary skill in the art as well as enhancers. The vector can be a commercially available preparation, such as an adenovirus vector (Quantum Biotechnologies, Inc. (Laval, Quebec, Canada).

As one example, vector delivery can be via a viral system, such as a retroviral vector system which can package a recombinant retroviral genome (see e.g., Pastan et al., Proc. Natl. Acad. Sci. U.S.A. 85:4486, 1988; Miller et al., Mol. Cell. Biol. 6:2895, 1986). The recombinant retrovirus can then be used to infect and thereby deliver to the infected cells nucleic acid encoding a broadly neutralizing antibody (or active fragment thereof) of the invention. The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naidini et al., Science 272:263-267, 1996), pseudotyped retroviral vectors (Agrawal et al., Exper. Hematol. 24:738-747, 1996). Physical transduction techniques can also be used, such as liposome delivery and receptor-mediated and other endocytosis mechanisms (see, for example, Schwartzenberger et al., Blood 87:472-478, 1996). This invention can be used in conjunction with any of these or other commonly used gene transfer methods.

In addition, the methods described for introducing nucleic acid and/or protein may be used to introduce NEDD4L into cells. Such introduction may be used to treat hypertension or inhibit viral budding. For example, a NEDD4L gene, or fragment thereof, may be introduced into cells. The NEDD4L gene or fragment may be integrated by illegitimate recombination or by homologous recombination. Alternatively, the NEDD4L gene or fragment may be maintained extrachromosomally, for example, as an episome.

As one example, if the antibody-encoding nucleic acid or some other nucleic acid encoding a protein recognizing the C2 domain of the NEDD4L gene product, NEDD4 gene product, or homologs and orthologs, or fragments of the same, gene product protein or encoding a particular variant of the NEDD4L gene, NEDD4 gene, or homologs and orthologs, or fragments of the same, to be used in the disclosed methods, is delivered to the cells of a subject in an adenovirus vector, the dosage for administration of adenovirus to humans can range from about 10⁷ to 10⁹ plaque forming units (pfu) per injection but can be as high as 10¹² pfu per injection (Crystal, Hum. Gene Ther. 8:985-1001,1997; Alvarez and Curiel, Hum. Gene Ther. 8:597-613,1997). A subject can receive a single injection, or, if additional injections are necessary, they can be repeated at six month intervals (or other appropriate time intervals, as determined by the skilled practitioner) for an indefinite period and/or until the efficacy of the treatment has been established.

Parenteral administration of the nucleic acid or vector of the present invention, if used, is generally characterized by injection. Injectables can be prepared in conventional forms, either as liquid solutions or suspensions, solid forms suitable for solution of suspension in liquid prior to injection, or as emulsions. Another approach for parenteral administration involves use of a slow release or sustained release system such that a constant dosage is maintained. See, e.g., U.S. Pat. No. 3,610,795, which is incorporated by reference herein. For additional discussion of suitable formulations and various routes of administration of therapeutic compounds, see, e.g., Remington: The Science and Practice of Pharmacy (19th ed.) ed. A. R. Gennaro, Mack Publishing Company, Easton, Pa. 1995.

Nucleic acids that are delivered to cells which are to be integrated into the host cell genome, typically contain integration sequences. These sequences are often viral related sequences, particularly when viral based systems are used. These viral integration systems can also be incorporated into nucleic acids which are to be delivered using a non-nucleic acid based system of deliver, such as a liposome, so that the nucleic acid contained in the delivery system can become integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring, for example, in vivo or in vitro.

Thus, the compositions can comprise, in addition to the disclosed compositions or vectors, for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci. USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc., Hilden, Germany) and TRANSFECTAM (Promega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochemica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6,399-409 (1991)).

NEDD4L containing a C2 domain is activated by phosphorylation and upon activation serves to target ENaC for receptor mediated endocytosis. Thus, NEDD4L containing a C2 domain serves to down regulate ENaC and decrease sodium absorption.

In vivo/ex vivo

As described above, the compositions can be administered in a pharmaceutically acceptable carrier and can be delivered to the subjects cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

Expression Systems

The nucleic acids that are delivered to cells typically contain expression controlling systems. For example, the inserted genes in viral and retroviral systems usually contain promoters, regulatory sequences and/or enhancers to help control the expression of the desired gene product. A promoter is generally a sequence or sequences of DNA that functions when operably linked to the transcription start site. A promoter contains core elements required for basic interaction of RNA polymerase and transcription factors, and may contain upstream elements and response elements.

Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g., beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729(1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription either positively (activation) or negatively (repression). Enhancers often determine the regulation of expression of a gene. Many enhancer sequences are now known from mammalian genes (globin, elastase, albumin, -fetoprotein and insulin), typically one will use an enhancer from a eukaryotic cell virus for general expression. For example, the SV40 enhancer on the late side of the replication origin (bp 100-270), the cytomegalovirus early promoter enhancer, the polyoma enhancer on the late side of the replication origin, and adenovirus enhancers may be used.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. A preferred promoter of this type is the CMV promoter (650 bases). Other preferred promoters are SV40 promoters, cytomegalovirus (fill length promoter), and retroviral vector LTF. In certain constructs the promoter and/or enhancer region may be present in all eukaryotic cell types, even if it is only expressed in a particular type of cell at a particular time.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that a polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences to improve expression from, or stability of, the construct.

Markers

The vectors of the invention can include nucleic acid sequence encoding a marker product. This marker product is used to determine if the gene has been delivered to the cell and may optionally be constructed such that expression of the gene is determined, for example, by constructing the marker and gene as a dicistronic message. Marker genes include, but are not limited to, the E. Coli lacZ gene, which encodes β-galactosidase, and green fluorescent protein.

In some embodiments the marker may be a selectable marker. Examples of suitable selectable markers for mammalian cells include, but are not limited to, dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media,

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

Pharmaceutical Carriers/Delivery of Pharmaceutical Products

As described herein, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like. As used herein, “topical intranasal administration” means delivery of the compositions into the nose and nasal passages through one or both of the nares and can comprise delivery by a spraying mechanism or droplet mechanism, or through aerosolization of the nucleic acid or vector. The latter may be effective when a large number of animals is to be treated simultaneously. Administration of the compositions by inhalant can be through the nose or mouth via delivery by a spraying or droplet mechanism. Delivery can also be directly to any area of the respiratory system (e.g., lungs) via intubation. The compositions may be delivered such that one or more organs, for example, the kidney, are preferentially targeted. Such organ targeting is known to a person of skill in the art and varies with the delivery vehicle. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

Pharmaceutically Acceptable Carriers

The compositions, including antibodies, can be used therapeutically in combination with a pharmaceutically acceptable carrier.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example, by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Therapeutic Uses

The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counter indications. Dosage can vary, and can be administered in one or more dose administrations daily, for one or several days.

Combinatorial Chemistry

The disclosed compositions can be used as targets for any combinatorial technique to identify molecules or macromolecular molecules that interact with the disclosed compositions in a desired way. Also disclosed are the compositions that are identified through combinatorial techniques or screening techniques in which the compositions interact with the NEDD4L gene product, NEDD4 gene product, or homologs and ortholog gene products or fragments of the same, such that the compositions specifically recognize the C2 domain, for example, where the compositions were identified using a NEDD4L gene product, NEDD4 gene product, or homologs and ortholog gene product or fragments of the same as targets in a screening or selection protocol.

It is understood that when using the disclosed compositions in combinatorial techniques or screening methods, molecules, such as macromolecular molecules, will be identified that have particular desired properties such as inhibition or stimulation or the target molecule's function. The molecules identified and isolated when using the disclosed compositions, such as, the NEDD4L gene product, NEDD4 gene product, or homologs and ortholog gene products or fragments of the same are used as targets, or when they are used in competitive inhibition assays are also disclosed. Thus, the products produced using the combinatorial or screening approaches that involve the disclosed compositions are also considered herein disclosed.

Combinatorial chemistry includes but is not limited to all methods for isolating small molecules or macromolecules that are capable of binding either a small molecule or another macromolecule, typically in an iterative process. Proteins, oligonucleotides, sugars, lipids, steroids and derivatives thereof, are examples of macromolecules. For example, oligonucleotide molecules with a given function, catalytic or ligand-binding, can be isolated from a complex mixture of random oligonucleotides in what has been referred to as “in vitro genetics” (Szostak, TIBS 19:89, 1992). One synthesizes a large pool of molecules bearing random and defined sequences and subjects that complex mixture, for example, approximately 10¹⁵ individual sequences in 100 μg of a 100 nucleotide RNA, to some selection and enrichment process. Through repeated cycles of affinity chromatography and PCR amplification of the molecules bound to the ligand on the column, Ellington and Szostak (1990) estimated that 1 in 10 RNA molecules folded in such a way as to bind a small molecule dyes. DNA molecules with such ligand-binding behavior have been isolated as well (Ellington and Szostak, 1992; Bock et al, 1992). Techniques aimed at similar goals exist for small organic molecules, proteins, antibodies and other macromolecules known to those of skill in the art. Screening sets of molecules for a desired activity whether based on small organic libraries, oligonucleotides, or antibodies is broadly referred to as combinatorial chemistry. Combinatorial techniques are particularly suited for defining binding interactions between molecules and for isolating molecules that have a specific binding activity, often called aptamers when the macromolecules are nucleic acids.

There are a number of methods for isolating proteins which either have de novo activity or a modified activity. For example, phage display libraries have been used to isolate numerous peptides that interact with a specific target. (See for example, U.S. Pat. Nos. 6,031,071; 5,824,520; 5,596,079; and 5,565,332 which are herein incorporated by reference at least for their material related to phage display and methods relate to combinatorial chemistry.)

A preferred method for isolating proteins that have a given function is described by Roberts and Szostak (Roberts R. W. and Szostak J. W. Proc. Natl. Acad. Sci. USA, 94(23)12997-302 (1997). This combinatorial chemistry method couples the functional power of proteins and the genetic power of nucleic acids. An RNA molecule is generated in which a puromycin molecule is covalently attached to the 3′-end of the RNA molecule. An in vitro translation of this modified RNA molecule causes the correct protein, encoded by the RNA to be translated. In addition, because of the attachment of the puromycin, a peptdyl acceptor which cannot be extended, the growing peptide chain is attached to the puromycin which is attached to the RNA. Thus, the protein molecule is attached to the genetic material that encodes it. Normal in vitro selection procedures can now be done to isolate functional peptides. Once the selection procedure for peptide function is complete traditional nucleic acid manipulation procedures are performed to amplify the nucleic acid that codes for the selected functional peptides. After amplification of the genetic material, new RNA is transcribed with puromycin at the 3′-end, new peptide is translated and another functional round of selection is performed. Thus, protein selection can be performed in an iterative manner just like nucleic acid selection techniques. The peptide which is translated is controlled by the sequence of the RNA attached to the puromycin. This sequence can be anything from a random sequence engineered for optimum translation (i.e., no stop codons, etc.) or it can be a degenerate sequence of a known RNA molecule to look for improved or altered function of a known peptide. The conditions for nucleic acid amplification and in vitro translation are well known to those of ordinary skill in the art and are preferably performed as in Roberts and Szostak (Roberts R. W. and Szostak J. W. Proc. Natl. Acad. Sci. USA, 94(23)12997-302 (1997)).

Another preferred method for combinatorial methods designed to isolate peptides is described in Cohen et al. (Cohen B. A., et al., Proc. Natl. Acad. Sci. USA 95(24):14272-7 (1998)). This method utilizes and modifies two-hybrid technology. Yeast two-hybrid systems are useful for the detection and analysis of protein:protein interactions. The two-hybrid system, initially described in the yeast Saccharomyces cerevisiae, is a powerful molecular genetic technique for identifying new molecules or fragments thereof, that specifically interact with the protein of interest (Fields and Song, Nature 340:245-6 (1989)). Cohen et al., modified this technology so that novel interactions between synthetic or engineered peptide sequences which bind a molecule of choice could be identified. The benefit of this type of technology is that the selection is done in an intracellular environment. The method utilizes a library of peptide molecules that attached to a transcription activation domain. A peptide of choice, for example, a portion of NEDD4L gene product, NEDD4 gene product, or homologs and ortholog gene products or fragment of the same is attached to a sequence specific DNA-binding domain of a transcriptional activation protein, such as Gal 4. By performing the two-hybrid technique on this type of system, molecules that interact with the desired fragments of the NEDD4L gene product, NEDD4 gene product, or homologs and ortholog gene products can be identified. The peptide of choice, the bait, may alternatively be fused to the transcription activation domain and the library, the prey, attached to the DNA-binding domain. Alternative screens based on the two-hybrid methodology have been developed and may be used in place of the traditional two-hybrid system. For example, Cdc25p is an essential gene in Saccharomyces cerevisiae and must be targeted to the plasma membrane to function. Therefore, conditional alleles of cdc25 are introduced into the desired strain and the strain propagated under permissive conditions. The assay is generally conducted under non-permissive conditions. The bait, or prey, may be fused to a membrane localization signal, such as a mystrilation site and the prey, or bait, may be fused to Cdc25p lacking the ability to be targeted to the plasma membrane. Protein-protein interaction between the bait and prey will complement the function of Cdc25 and restore viability.

Using methodology well known to those of skill in the art, in combination with various combinatorial libraries, one can isolate and characterize those small molecules or macromolecules, which bind to or interact with the desired target. The relative binding affinity of these compounds can be compared and optimum compounds identified using competitive binding studies, which are well known to those of skill in the art.

Techniques for making combinatorial libraries and screening combinatorial libraries to isolate molecules which bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636.

Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. Nos. 6,017,768 and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (U.S. Pat. No. 5,856,107) substituted 2-methylene-2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits (U.S. Pat. Nos. 5,698,685 and 5,506,337), sulfamides (U.S. Pat. No. 5,618,825), and benzodiazepines (U.S. Pat. No. 5,288,514).

As used herein combinatorial methods and libraries included traditional screening methods and libraries as well as methods and libraries used in iterative processes.

Computer Assisted Drug Design

The disclosed compositions can be used as targets for any molecular modeling technique to identify either the structure of the disclosed compositions or to identify potential or actual molecules, such as small molecules, which interact in a desired way with the disclosed compositions.

It is understood that when using the disclosed compositions in modeling techniques, molecules, such as macromolecular molecules, will be identified that have particular desired properties such as inhibition or stimulation or the target molecule's function. The molecules identified and isolated when using the disclosed compositions are also disclosed. Thus, the products produced using the molecular modeling approaches that involve the disclosed compositions are also considered herein disclosed.

Chips and Micro Arrays

Disclosed are chips where at least one address is the sequences or part of the sequences set forth in any of the nucleic acid sequences disclosed herein. Also disclosed are chips where at least one address is the sequences or portion of sequences set forth in any of the peptide sequences disclosed herein.

Also disclosed are chips where at least one address is a variant of the sequences or part of the sequences set forth in any of the nucleic acid sequences disclosed herein. Also disclosed are chips where at least one address is a variant of the sequences or portion of sequences set forth in any of the peptide sequences disclosed herein.

Disclosed are chips where at least one address is a molecule related to the variant 13 position as disclosed herein. For example, a chip comprising sequence flanking the variant 13 position are disclosed. Also disclosed are chips comprising any of the sequence information set forth in Table 3.

Computer Readable Mediums

It is understood that the disclosed nucleic acids and proteins can be represented as a sequence consisting of the nucleotides of amino acids. There are a variety of ways to display these sequences, for example, the nucleotide guanosine can be represented by G or g. Likewise the amino acid valine can be represented by Val or V. Those of skill in the art understand how to display and express any nucleic acid or protein sequence in any of the variety of ways that exist, each of which is considered herein disclosed. Specifically contemplated herein is the display of these sequences on computer readable mediums, such as, commercially available floppy disks, tapes, chips, hard drives, compact disks, and video disks, or other computer readable mediums. Also disclosed are the binary code representations of the disclosed sequences. Those of skill in the art understand what computer readable mediums. Thus, computer readable mediums on which the nucleic acids or protein sequences are recorded, stored, or saved.

Disclosed are computer-readable mediums comprising the sequences and information regarding the sequences set forth herein. Also disclosed are computer-readable mediums comprising the sequences and information regarding the sequences set forth herein.

Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended. For example, disclosed is a kit for assessing a subject's risk for acquiring hypertension, comprising primers capable of identifying sequence information at the variant 13 position, as disclosed herein, of a particular subject or DNA sample. Also disclosed are kits containing reagents as discussed herein related to any of the sequences set forth in Table 3.

Also disclosed are kits containing reagents for assessing the presence, absence or amount of NEDD4L having a C2 domain and/or NEDD4L lacking the C2 domain.

Methods of Making the Compositions

The compositions disclosed herein and the compositions necessary to perform the disclosed methods can be made using any method known to those of skill in the art for that particular reagent or compound unless otherwise specifically noted.

Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see, for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1 Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

Peptide Synthesis

One method of producing the disclosed proteins is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide, W. H. Freeman and Co., N.Y. (1992); Bodansky M and Trost B., Ed. (1993) Principles of Peptide Synthesis, Springer-Verlag Inc., NY (which is herein incorporated by reference at least for material related to peptide synthesis).) Alternatively, the peptide or polypeptide is independently synthesized in vivo as described herein. Once isolated, these independent peptides or polypeptides maybe linked to form a peptide or fragment thereof via similar peptide condensation reactions.

The peptides may also be directly or indirectly linked to other molecules, such as, fluorescent molecules, biotin, enzymes or the like. In addition, one or more peptides maybe labeled by incorporation of radioactive isotopes. For example, peptides may be labeled by methods known in the art for incorporating ¹²⁵I, ¹⁴C or ³H.

For example, enzymatic ligation of cloned or synthetic peptide segments allow relatively short peptide fragments to be joined to produce larger peptide fragments, polypeptides or whole protein domains (Abrahmsen L. et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation., Science, 266:776-779 (1994)). The first step is the chemoselective reaction of an unprotected synthetic peptide-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M. et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV, Academic Press, New York, pp. 257-267 (1992)).

Processes for Making the Compositions

Disclosed are processes for making the compositions as well as making the intermediates leading to the compositions. For example, disclosed are process for making primers and probes and kits. There are a variety of methods that can be used for making these compositions, such as synthetic chemical methods and standard molecular biology methods. It is understood that the methods of making these and the other disclosed compositions are specifically disclosed.

Disclosed are cells produced by the process of transforming the cell with any of the disclosed nucleic acids. Disclosed are cells produced by the process of transforming the cell with any of the isolated nucleic acids disclosed herein.

Disclosed are any of the disclosed peptides produced by the process of expressing any of the disclosed nucleic acids. Disclosed are any of the isolated peptides produced by the process of expressing any of the disclosed nucleic acids.

Disclosed are animals produced by the process of transfecting a cell within the animal with any of the nucleic acid molecules disclosed herein. Disclosed are animals produced by the process of transfecting a cell within the animal with any of the nucleic acid molecules disclosed herein, wherein the animal is a mammal. Also disclosed are animals produced by the process of transfecting a cell within the animal with any of the nucleic acid molecules disclosed herein, wherein the mammal is mouse, rat, rabbit, cow, sheep, pig, or primate.

Also disclosed are animals produced by the process of adding to the animal any of the cells disclosed herein.

Methods of Using the Compositions

Methods of Using the Compositions as Research Tools

The disclosed compositions can be used in a variety of ways as research tools. For example, the disclosed compositions, can be used to study the interactions between NEDD4L gene product, NEDD4 gene product, or homologs and ortholog gene products and, for example, ENaC.

The compositions can be used, for example, as targets in combinatorial chemistry protocols or other screening protocols to isolate molecules that possess desired functional properties related to, for example, the C2 domain, and molecules which can be used to identify variants of the NEDD4L gene product, NEDD4 gene product, or homologs and ortholog gene products.

The disclosed compositions can be used as discussed herein as either reagents in micro arrays or as reagents to probe or analyze existing microarrays. The disclosed compositions can be used in any known method for isolating or identifying single nucleotide polymorphisms. The compositions can also be used in any method for determining allelic analysis of, for example, the NEDD4L gene, NEDD4 gene, or homologs and orthologs, or fragments of the same, particularly allelic analysis as it relates to the variant 13 position of the NEDD4L gene or those positions identified Table 3. The compositions can also be used in any known method of screening assays, related to chip/micro arrays. The compositions can also be used in any known way of using the computer readable embodiments of the disclosed compositions, for example, to study relatedness or to perform molecular modeling analysis related to the disclosed compositions.

Methods of Diagnosing or Predicting Hypertension

The disclosed compositions can also be used as diagnostic tools related to diseases, such as, hypertension and viral infectivity. Disclosed are methods of determining a subject's risk for acquiring hypertension, comprising assaying whether the subject has an A or G at the variant 13 position of the NEDD4L gene. Also disclosed are methods of determining a subject's risk for acquiring hypertension, comprising assaying whether the subject has any of the variations set forth in Table 3.

The steps of assaying subjects or DNA samples for the variations of the NEDD4L gene, NEDD4 gene, or homologs and orthologs, or fragments of the same, discussed herein, would include, but are not limited to, collecting the nucleic acid sample, such as DNA, by, for example, collecting cells from a subject and isolating the desired nucleic acid in any way desired. The disclosed methods could also include steps, such as amplifying the nucleic acid, separating the nucleic acid, purifying the nucleic acid, categorizing the nucleic acid in any way desired. The disclosed methods could also include hybridization steps using, for example, chip technology. Typically the method will also include a step of identifying the sequence or variation present in the sample nucleic acid related to, for example, variant 13 of NEDD4L or any of the variants discussed in Table 3. The variants of Table 3 all show linkage to other variants disclosed in Table 3, therefore, information regarding one variant correlates with the presence of other variants. The method may also include steps related to the analysis and quantitation of the information obtained from the analysis of the sample. This type of analysis can be performed by, for example, computers.

Methods of Gene Modification and Gene Disruption

The disclosed compositions and methods can be used for targeted gene disruption and modification in any animal that can undergo these events. Gene modification and gene disruption refer to the methods, techniques, and compositions that surround the selective removal or alteration of a gene or stretch of chromosome in an animal, such as a mammal, in a way that propagates the modification in the target cells and optionally through the germ line of the mammal. In general, a cell is transformed with a vector which is designed to homologously recombine with a region of a particular chromosome contained within the cell, as, for example, described herein. This homologous recombination event can produce a chromosome which has exogenous DNA introduced, for example, in frame, with the surrounding DNA. This type of protocol allows for very specific mutations, such as point mutations, to be introduced into the genome contained within the cell. Methods for performing this type of homologous recombination are disclosed herein.

One of the preferred characteristics of performing homologous recombination in mammalian cells is that the cells should be able to be cultured, because the desired recombination event occurs at a low frequency.

Once the cell is produced through the methods described herein, an animal can be produced from this cell through either stem cell technology or cloning technology. For example, if the cell into which the nucleic acid was transfected was a stem cell for the organism, then this cell, after transfection and culturing, can be used to produce an organism which will contain the gene modification or disruption in germ line cells, which can then in turn be used to produce another animal that possesses the gene modification or disruption in all of its cells. In other methods for production of an animal containing the gene modification or disruption in all of its cells, cloning technologies can be used. These technologies generally take the nucleus of the transfected cell and either through fusion or replacement fuse the transfected nucleus with an oocyte which can then be manipulated to produce an animal. The advantage of procedures that use cloning instead of ES technology is that cells other than ES cells can be transfected. For example, a fibroblast cell, which is very easy to culture can be used as the cell which is transfected and has a gene modification or disruption event take place, and then cells derived from this cell can be used to clone a whole animal. Where a clonal animal is to be produced, the animal may be any animal other than a human, such as, a mouse, rat, rabbit, primate or the like.

Disclosed, for example, would be animals having a gene disruption event, taking place at, for example, the variant 13 position, or the positions identified in Table 3, for the purpose of creating, for example, an animal model system for the study of hypertension or for testing drugs against hypertension.

Methods of Treating

Disclosed are compositions and methods for treating hypertension. Disclosed is the relationship between the C2 domain of NEDD4L and NEDD4L homologs and orthologs, or fragments of the same, and the Na transport enzyme ENaC. It is found that promoting this interaction can reduce hypertension.

Also disclosed are compositions and methods for preventing viral budding. Disclosed is the relationship between the C2 domain of NEDD4L and NEDD4L homologs and orthologs, or fragments of the same, and the viral building machinery. It is found that promoting this interaction can reduce viral budding.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### Material and Methods

Sequence Analysis

Genomic, cDNA and EST databases included GenBank nr, Human and Mouse EST entries, the human genome draft assembly hg8 06 Aug. 2001 freeze (http://genome.ucsc.edu), the mouse phusion and arachne draft assembly (http://mouse.ensembl.org) and the Fugu rubripes draft genome assembly 1 (http://wwwjgi.doe.gov/fugu/index.html). Results from BLAST and cross-match analysis were parsed with Perl scripts that iterated the sequence search to form a consistent assembly of EST, cDNA and genomic sequence. Inclusion of EST and cDNA sequence into the assembly required at least one confirmed splice junction on genomic DNA. All genomic coordinates are referenced to the chromosome 18 draft assembly hg8 06 Aug. 2001 freeze or contig NT_{—}033907, gi=27485382. The coordinates reported in relation to chromosome 18 draft assembly hg8 06 Aug. 2001 freeze are indexed to base 65,000,000 in that assembly.

Genomic DNA used for Polymorphism Analysis

Hypertensive sibpairs and normotensive controls for the investigation of genetic linkage to a wide variety of phenotypes (Williams et al. 2000). DNA samples were selected from 50 normotensive controls for this survey of common polymorphism. DNA samples were selected from over 430 hypertensive patients and surveyed for the polymorphisms shown in Table 3. All participants were hypertensive (systolic blood pressure≧140 mm Hg, diastolic blood pressure≧90 mm Hg, or on antihypertensive medications) with diagnosis before age 60.

Search for Polymorphism by Resequencing

PCR amplification was carried out in 50 μl reaction volumes using Expand Long Template PCR System (Roche); see supplementary data for primer sequences. Each reaction contained 100 ng of genomic DNA, 350 μM dNTPs, 0.2 μM of each PCR primer, 1× reaction buffer #2 and 2.6 units of Taq/Pwo polymerase mix. Cycling conditions included an initial denaturation at 94° C. for 2 min., 10 cycles of 94° C. for 10 sec., 55° C. for 10 sec., 68° C. for 2 min.; followed by 20 cycles of 94° C. for 10 sec., 55° C. for 10 sec., 68° C. for 2 min.+20 sec./cycle. Residual primers and dNTPs were removed from PCR products with a Millipore glass fiber filter. The sequence-ready templates were eluted in 70 μl of sterile H₂O. 5 μl of each template was aliquoted to a 384-well sequence dish and evaporated to dryness in a speed-vac.

Cycle sequencing was carried out in 2 μl reaction volumes using ABI BigDye Terminator v.3.0 chemistry. Cyc° C. for 10 sec., 50° C. for 5 sec., 60° C. for 4 min. Upon completion of cycle sequencing, ling conditions included an initial denaturation at 96° C. for 30 sec.; followed by 45 cycles of 96 8 μl of 62.5% EtOH/1M KOAc pH4.5 was added to each reaction and the sequence plates were centrifuged at 4000 rpm at 4° C. for 45 min. The samples were resuspended in 10 μl of formamide and electrophoresed on an ABI 3700 DNA analyzer prepared with POP-5 capillary gel matrix. Sequence trace files were evaluated using the Phred, Phrap and Consed programs (Ewing et al. 1998). Potential heterozygotes were identified by using the PolyPhred version 3.5 program (Nickerson et al. 1997). Polymorphisms were verified by manual evaluation of the individual sequence traces.

Reverse Transcription-PCR (RT-PCR) and Rapid Amplification of cDNA Ends (RACE)

5′ RACE reactions were performed with the SMART II RACE cDNA amplification kit (Clontech, USA) according to the manufacturer's protocol. Total RNA was purchased from Clontech (Catalog #: 64096-1, 64097-1) and Stratagene (Catalog #: 735014, 735474). First strand cDNA synthesis was performed with PowerScript reverse transcriptase (Clontech, USA) on 1 μg of total RNA using the 5′ RACE CDS primer and the addition of SMART II A oligonucleotide (Clontech, USA). Following reverse transcription, the first-strand cDNA is used directly in 5′ RACE or RT-PCR reactions. Gene specific PCR products were obtained by using a nested PCR strategy (see supplemental data for primer sequences). Products were sub-cloned into pCR2.1 with the TA Cloning Kit (Invitrogen, USA). Standard plasmid sequencing procedures were performed on the sub-clones and RT-PCR templates were also sequenced directly.

Quantitative Analysis of mRNA

Human kidney, adrenal gland and liver mRNA, purchased from Clontech, were used for quantitative analysis of mRNA. First-strand cDNA for 1 μg total RNA was prepared using reverse transcriptase (Power Script Reverse Transcriptase, Clontech) using the manufacturer's standard protocol. Quantitative real-time polymerase chain reaction (Q-PCR) was performed by monitoring the fluorescence of SYBR Green (Molecular Probes) with the ABI PRISM 7700 sequence detection system (Applied Biosystems). Oligonucleotide primers were designed to span at least 1 intron and to minimize primer-dimer formation. PCR reaction products were electrophoresed to verify specific amplification and the absence of primer-dimer formation. All PCR reactions were performed in triplicate with primers specific for the corresponding human genes and spanning at least 1 intron: 5′ cct aaa tga gac gtc tcg cat ttg ag 3′ (human N4L Ex1 up), 5′ agc tgg cgg aga cca gga ttt 3′ (human NEDD44L Ex2a UP), 5′ ccg cta cgt aca atg aaa gtt tca c 3′(human NEDD4L Ex3 RP), 5′gaa ggt gaa ggt cgg agt c 3′ (human glyceraldehyde-3-phosphate dehydrogenase (hGAPDH) UP), 5′ gaa gat ggt gat ggg att tc 3′ (human GAPDH RP). Amplification was performed during 35 cycles of 94° C. for 10 seconds, 60° C. for 10 seconds, and 72° C. for 20 seconds. Water and genomic DNA served as negative controls. Cloned human cDNAs were used to generate standard curves. Expression of both Ex1-Ex3 isoform and Ex2a-Ex3isoform was expressed relative to hGAPDH expression.

### Example 2

### Results

Polymorphism Discovery in NEDD4L

Common variants of human NEDD4L were discovered by sequencing PCR products spanning exons 1, 2, 2a and 3 through 31 from 48 individuals. Exons 1 and 2 were defined from EST sequences (BF965237 (SEQ ID NO:152) and BF678906 (SEQ ID NO:153)) and TBLASTN analysis of genomic sequence. Exon 2a corresponds to the splice form typified by KIAA0439 (GenBank accession no. ABO07899 (SEQ ID NO: 154)). The location of NEDD4L exon junctions and the target coordinates for resequencing are in Table 3. 5,524 nucleotides in exons and 23,108 nucleotides overall were surveyed for polymorphisms by resequencing. Table 3 shows the position, alleles, allele frequency and functional implication of the 34 single nucleotide and 4 insertion/deletion polymorphisms detected. One polymorphism, variant 13 at nucleotide 82,723, occurred at the last nucleotide of exon 1 and is common in Caucasians (70% G, 30% A.). This variant has the potential to disrupt exon 1 splicing since G is the most common nucleotide at this position in consensus 5′ splice donor sites (Stephens and Schneider 1992). Variants in this position are known to alter splice site selection in numerous human mutations (Nakai and Sakamoto 1994; Rogan et al. 1998). Splice site selection at exon 1 was evaluated in human kidney and adrenal RNA.

Transcript Analysis by RT-PCR, RACE and Q-PCR

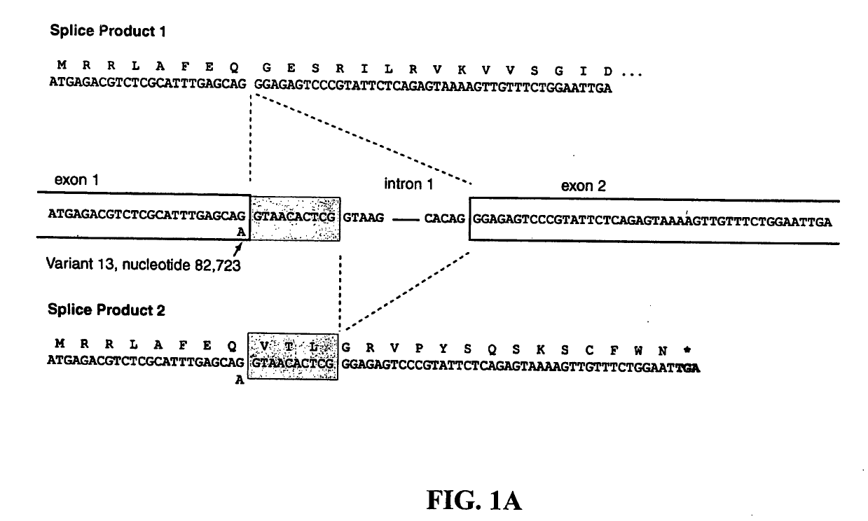

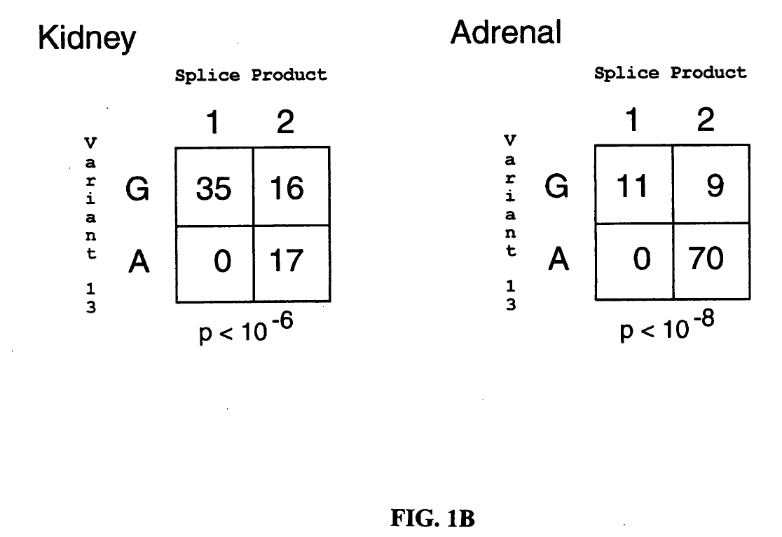

RT-PCR was performed with exon 1 and exon 3 specific primers. Two sources of RNA from each tissue were used: kidney 1=normal, whole kidneys pooled from 6 male/female Caucasians, kidney 2=2 female donors, adrenal 1=normal, whole adrenal glands pooled from 62 male/female Caucasians, adrenal 2=single female. Direct sequencing of the RT-PCR products from kidney RNA displayed mixed sequence at the exon 1-2 junction, so the products were sub-cloned into a plasmid vector, and independent transformants were sequenced. FIG. 1 shows that two distinct splice junctions are found: splice product 1 generates an intact predicted reading frame between exon 1 and 2, while splice product 2, generated by splicing 10 nts. distal of splice product 1, results in a transcript that disrupts the predicted reading frame. The G variant shows leaky splice site selection with a mixture of splice product 1 (35/51 in kidney, 11/20 in adrenal) and splice product 2 (16/51 in kidney, 9/20 in adrenal), while the A variant shows only splice product 2 (87/87 in kidney and adrenal).

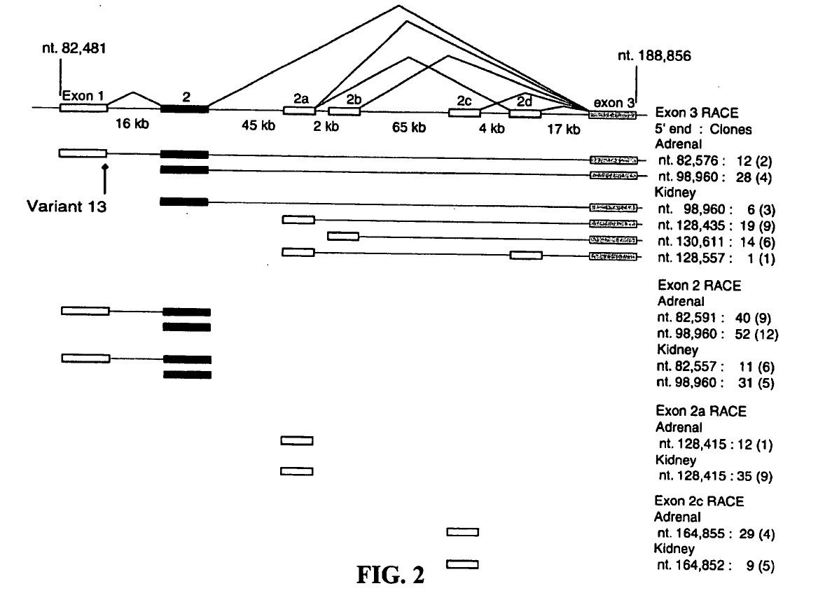

Further characterization of the representation and semi-quantitative abundance of 5′ splice isoforms were performed with 5′ RACE reactions with primers specific for exons 2, 2a, 2c and 3, using the same 4 RNA preparations used in the RT-PCR experiment. RACE products were sub-cloned into a plasmid vector and 96 clones were sequenced for each RACE reaction. FIG. 2 shows the exon structure of the RACE products and the number of unique 5′ ends seen with each primer. Using primers within exon 2, approximately 50% of the sub-cloned RACE products from kidney and adrenal showed splicing to exon 1. The other products were located in exon 2 or spliced to unrepresented genomic sequence. Race experiments with both exon 2a and 2c primers resulted in no detectable 5′ sequence spliced to these exons, implying that these exons may be adjacent to promoters expressed in these tissues. RACE experiments with exon 3 primers confined the presence of the exon 1-exon 2-exon 3 (isoform I) transcript, and the exon 2a-3 (isoform II) transcript. These experiments also revealed two distinct exons expressed in kidney: 2b and 2d, with 2b represented as a 5′ exon without an in-frame AUG codon spliced to exon 3, similar to exon 2a and 2c, and exon 2d detected as an alternative splice form consisting of exon 2a-2d-3, each with consensus splice sequences at their 5′ donor and 3′ acceptor sites. The exon 1-2 splice junction for RACE clones containing the G variant were either splice product 1 or 2, and only splice product 2 was seen when the A variant was present, similar to the RT-PCR analysis.

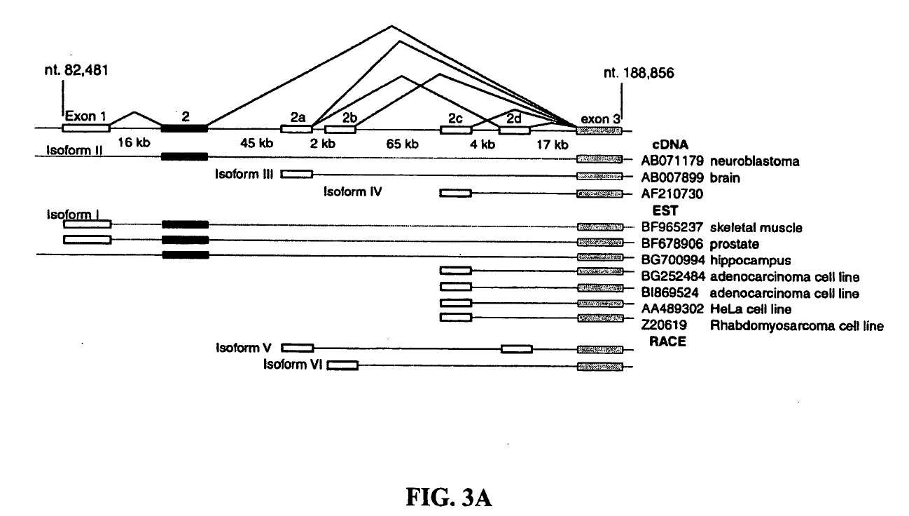

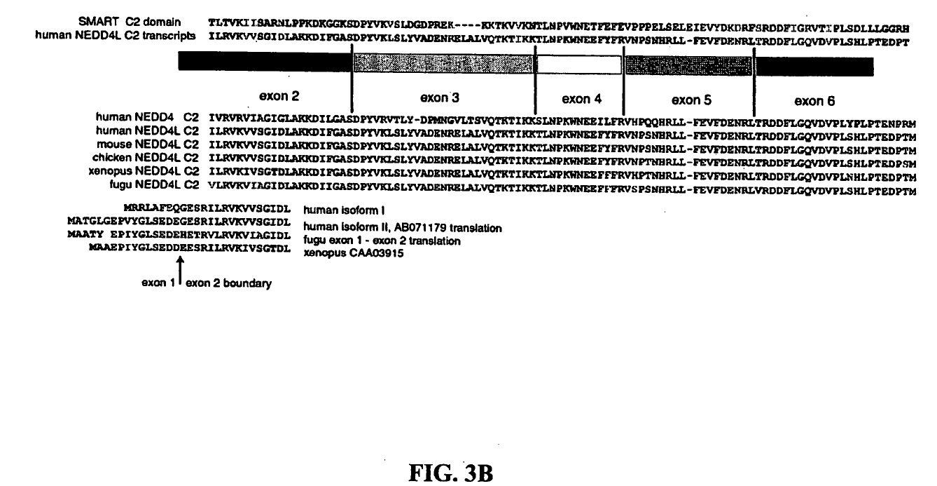

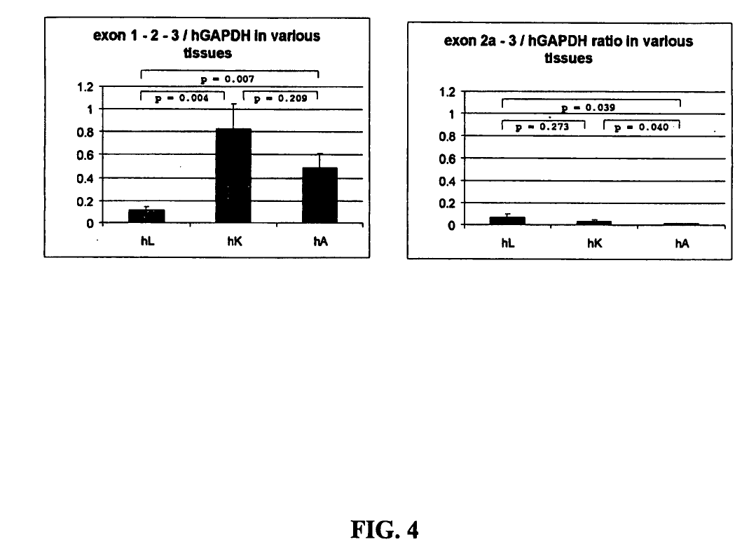

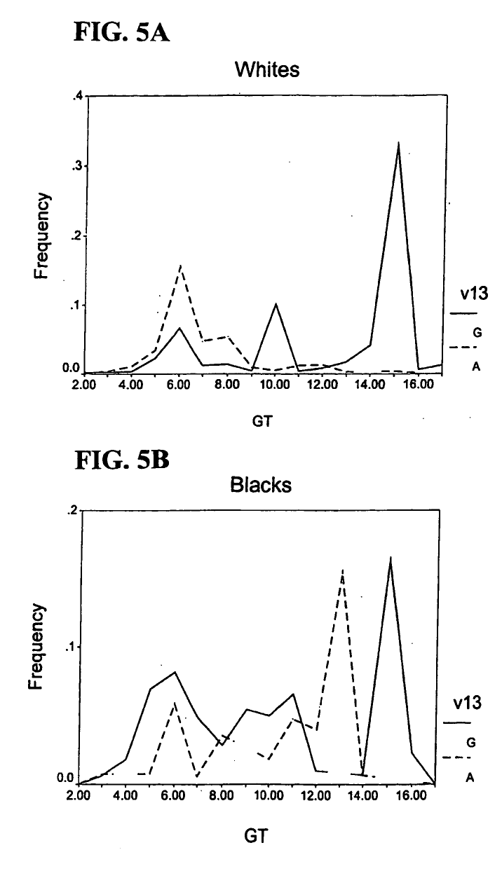

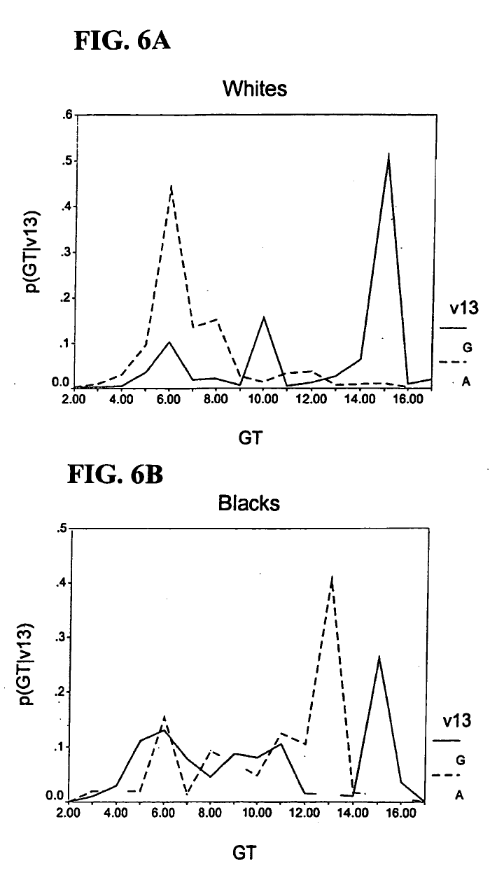

The relative level and tissue specific expression of the exon 1-2-3 isoform I versus the exon 2a-3 isoform III was analyzed by Q-PCR. FIG. 3 shows the results of 4 experiments, each in triplicate, for the quantitative analysis of NEDD4L mRNA from kidney, adrenal gland and liver, normalized to the expression of human GAPDH in each tissue. Expression of GAPDH in these tissues is not significantly different (data not shown). Expression of isoform I is significantly higher in both kidney and adrenal than isoform III. These findings suggest that isoform I (exon 1-2-3) is expressed in a tissue-specific manner, and that is more abundant than isoform III (exon 2a-3) in kidney and adrenal gland.

Definition of NEDD4L Genomic Structure and Transcript Isoform Pattern

FIG. 3A combines cDNA, EST, RT-PCR and RACE data and suggests 6 different transcript isoforms spliced to exon 3. Isoforms I and II splice an in-frame AUG codon to exon 2 adding 8 and 16 amino acids, respectively. Isoform I spans 303 kb of genomic DNA (draft sequence with 54 gaps) and consists of 30 exons. Both isoforms are capable of producing a translated protein now containing an evolutionarily conserved N-terminal C2 domain beginning in exon 2. The similarity of this C2 domain to consensus SMART C2 domain sequence (Schultz et al. 2000) is shown in FIG. 3B, as well as the location of this domain across exons 2 through exon 6 of NEDD4L. The sequence 5′ of the exon 2 splice junction in isoform II is not represented in the current version of the draft human genome sequence, and we postulate it may reside 5′ of exon 1, in an unfinished region of chromosome 18. This isoform was not detected in the 5′ RACE analysis, but is detected by RT-PCR from both kidney and adrenal RNA (data not shown).

Isoforms III and IV splice exons 2a and 2c to exon 3, respectively. Most functional studies of human NEDD4L protein have used the exon 2a-3 isoform typified by KIAA0439 (GenBank accession no. ABO07899 (SEQ ID NO:154)), which lacks an intact N-terminal C2 domain. Neither of these exons contain AUG codons in any frame, and the first AUG start codon in these isoforms occurs in exon 7, thus producing a form of NEDD4L protein completely lacking the C2 domain. Isoforms V (exon 2a-2d-3) and VI (exon 2d-3) also lack AUG start codons and are predicted to initiate translation in exon 7.

Analysis of mouse cDNA, EST and draft genomic assemblies reveals conserved exon-intron junctions for exons 2, 2a, 2b, 2c and 3. Mouse exon 2a-3 splice form is represented by the following ESTs: AW106584 (SEQ ID NO:155), BI687556 (SEQ ID NO: 156), BB621848 (SEQ ID NO:157), AW228518 (SEQ ID NO:158), BI218843 (SEQ ID NO:159), AW226920 (SEQ ID NO: 160), BB846219 (SEQ ID NO: 161), AI527754 (SEQ ID NO: 162), AI227149 (SEQ ID NO: 163), AI931594 (SEQ ID NO:164), AW910412 (SEQ ID NO:165), BI690367 (SEQ ID NO:166), BG971715 (SEQ ID NO: 167), BI650832 (SEQ ID NO: 168), BI559036 (SEQ ID NO: 169). Mouse exon 2b-3 splice isoform is represented by cDNA AKO04969 (SEQ ID NO: 170), and mouse exon 2c splice isoform is represented by ESTs: BG404747 (SEQ ID NO:171) and BG294676 (SEQ ID NO: 172). The mouse exon 2-3 splice form is represented by ESTs: BB569841 (SEQ ID NO: 173), BB611456 (SEQ ID NO:174), BB635343 (SEQ ID NO:175), BB639757 (SEQ ID NO:176), BB640919 (SEQ ID NO: 177), BB847602 (SEQ ID NO: 178), BB863379 (SEQ ID NO: 179). These seven ESTs form 5 sequence clusters, and two of these clusters match draft assemblies with a consensus GT dinucleotide at the predicted 5″ splice donor site. None of these clusters display significant similarity to human exon 1, although BB863379 (SEQ ID NO: 179) does have 71 nts. 5′ of exon 2 that matches the 5′ end of human isoform II at 89% identity. However this EST is only 96% identical to the other mouse exon 2-3 ESTs, thus it is likely that this EST represents a pseudogene.

TBLASTN analysis of isoform I and II against the draft Fugu rubripes (puffer fish) genome revealed a draft assembly (Scaffold_{—}826, 55 kb with 9 gaps) spanning 26 kb containing exons 2 through 30 (exons 10, 11, 12 are missing from the assembly) and displaying conserved exon-intron junctions for the 26 exons detected. Translation of the fugu NEDD4L exons yields a protein that is 87% identical and 94% similar to human NEDD4L isoform II. FIG. 3B shows that there are no amino acid substitutions between the C2 domain of human and mouse NEDD4L, and that there are fewer substitutions between xenopus, fugu, chicken, mouse and human NEDD4L than between human NEDD4L and NEDD4. The fugu NEDD4L gene also contains an exon 1.2 kb upstream of exon 2 that is predicted to splice an in-frame AUG codon to exon 2 with the addition of 15 N-terminal amino acids. That translated product displays detectable sequence similarity with the translated product of isoform II (FIG. 3B), which is the isoform whose sequence 5′ of exon 2 is missing in current human finished and draft sequence databases. Although orthologous 2a, 2b and 2c exons are detected in the mouse draft sequence, there is no detectable similarity in fugu NEDD4L intron 2 using either TBLASTN or BLASTN analysis. This suggests that isoform II may reflect the most ancestral form of NEDD4L and that the sequence of the N-terminal C2 domain has been conserved by natural selection.

The survey of NEDD4L exons and flanking introns for polymorphic variants in 48 Caucasian individuals detected 35 flanking/intronic variants, and 3 exonic variants. The survey of NEDD4L exons and flanking introns in over 430 patients diagnosed with hypertension, confirming the survey results in Caucasians. Variant 13-A occurs at an allele frequency of 0.30, and its location in exon 1 results in a synonymous CAG/A glutamine codon substitution, but also a change from the most common 5′ splice donor consensus to weaker consensus: CAG-GT to CAA-GT. Analysis of the exon 1-2 splice junction from RT-PCR and RACE products from human and adrenal RNA reveals leaky splice site selection, of which only one form is predicted to lead to in-frame translation of an evolutionarily conserved N-terminal C2 domain. The identity of variant 13 changes the ratio of splice site selection from preferred splicing of the in-frame product with the G variant, to no detectable splicing of the in-frame product with the A variant.

RT-PCR and RACE analysis from human and adrenal RNA revealed six transcript isoforms. Most functional studies of NEDD4L protein have used a spliced form of the human NEDD4L protein typified by KIAA0439 (GenBank accession no. AB007899 (SEQ ID NO: 154)), which corresponds to isoform III resulting from exon 2a-3 splicing and lacking an intact N-terminal C2 domain. This is in contrast to the founding, and defining, member of the NEDD4L family, a Xenopus laevis Nedd4-like protein that does contain a C2 domain (Rebhun and Pratt 1998). The analysis disclosed herein of genomic, cDNA and EST sequence reveals a conserved exon 2 in fugu, mouse and human that correspond to the orthologous sequence in xenopus. The RACE analysis using exon 2 and exon 3 primers demonstrates that isoform I of human NEDD4L is an abundant 5′ transcript isoform in adrenal tissue, while in kidney there is evidence for multiple isoforms including I, III, V and VI. Isoforms III through VI all generate transcripts where the first potential AUG start codon is in exon 7, distal to the C2 domain. RACE experiments from exon 2a and 2c did not detect any sequence spliced 5′ of these exons, and exon 1-3 RT-PCR revealed only exon 1-2-3 splice forms, with no splicing detected to exons 2a, 2b, 2c or 2d. Therefore, these isoforms are expected to result in translation from the AUG codon in exon 7. This methionine codon is conserved between human, mouse, chicken, xenopus and fugu, and as shown in FIG. 3B, it is located at the C-terminal junction of the C2 domain.

The observed average amino acid identity observed in large-scale comparisons of orthologous mouse and human protein sequences is 85.4% (Makalowski et al. 1996). The translated 101 amino acid C2 domain of human and mouse NEDD4L are 100% identical, 98.0% identical to chicken C2, 96.0% identical to xenopus C2, and 94.1% identical to fugu C2 domains. This level of sequence conservation suggests that the C2 domain is functional and under strong selective constraints. The existence of multiple isoforms that result in either C2 or C2-less forms of NEDD4L protein suggests that these two forms may have different functional roles in ubiquitination and endocytosis of protein targets such as ENaC.

Although no experiments have yet been reported with a human or mouse NEDD4L protein containing an intact C2 domain from either isoform I or II, there are several studies using C2-less NEDD4L and Nedd4/Rsp5 proteins in mammalian, amphibian and yeast systems. The approximately 110 amino acid C2 domain is a eukaryotic protein module that has Ca²⁺ dependent interactions with phospholipids, inositol polyphosphates and intracellular proteins. The human Nedd4 C2 domain has been shown to cause Ca²⁺ dependent plasma membrane localization in polarized Madin-Darby canine kidney cells (Plant et al. 1997). This localization of endogenous Nedd4 was preferably to apical and lateral membranes in these polarized cells, and heterologous expression of a C2 deletion construct showed no evidence of Ca²⁺ dependent membrane localization.

Further studies have shown that annexin XIlla and b is the protein-binding partner of Nedd4 in this Ca²⁺ dependent membrane localization assay (Plant et al. 2000). The Ca²⁺ dependent binding to annexin Xlllb further targets the complex to lipid rafts, which are membrane cholesterol and sphingolipid microdomains involved in endocytosis (Ikonen 2001).

Several experiments have investigated the effect of the Nedd4 C2 domain on regulation of ENaC activity. In both Xenopus oocytes and rat thyroid epithelia, removal of the Nedd4 C2 domain in expression constructs caused a stronger down-regulation of ENaC activity (Snyder et al. 2001). This result has also been seen in a comparison of human NEDD4 and NEDD4L in down-regulating ENaC activity in Xenopus oocytes (Kamynina et al. 2001a; Kamynina et al. 2001b). In this case, removal of Nedd4 C2 domain also resulted in stronger down-regulation of ENaC activity, and expression of an isoform III construct of NEDD4L (KIAA0439, SEQ ID NO: 154) displayed more potent inhibitor of ENaC than NEDD4 constructs.

Experiments with the RSP5 yeast homolog of the NEDD4/NEDD4L family have demonstrated that it is involved in the ubiquitination and endocytosis of several membrane proteins. C2 less constructs of Rsp5 had no effect on the ubiquitination and internalization of the yeast alpha-factor receptor. However, the C2 less construct displayed defects in transport of fluid-phase markers through the endocytotic pathway to the vacuole, suggesting that the C2 domain may have functions post-endocytosis suggesting that Nedd4 proteins are internalized along with their ubiquitinated protein targets (Dunn and Hicke 2001). This study also provided evidence that Rsp5 protein forms homomeric complexes, both in vitro and in vivo.

Recent evidence suggests that Nedd4 family proteins interact with coat proteins of enveloped viruses late in the process of viral budding (Vogt 2000; Hicke 2001). Using the late assembly domain of Rous sarcoma virus (RSV) Gag protein as a peptide probe on a chicken embryo expression library has recently resulted in the cloning of the C2 and WW domains of the chicken NEDD4L ortholog (Kikonyogo et al. 2001). In a human cell assay of RSV Gag budding, over-expression of the chicken NEDD4L WW domains resulted in dominant-negative inhibition of Gag budding, suggesting the over-expressed chicken NEDD4L WW domains inhibited Gag budding by competing with endogenous human NEDD4L. It has also been shown that the PY domain of ENaC can substitute for the RSV Gag late assembly domain in a virus-like particle assay in HeLa cells (Strack et al. 2000) implicating NEDD4L function in the budding of enveloped viruses containing PY motifs which include retroviruses.

In summary, a common variant in NEDD4L has been shown to affect splice site selection in a major transcript isoform expressed in kidney and adrenal gland. The variety of transcripts detected in human and mouse leading to either inclusion or exclusion of the C2 domain suggest that expression of different isoforms may lead to functional differences. Indeed, presence of the C2 domain, by targeting such isoforms to cell membranes, may contribute to substrate specificity and the proposed interactions with ENaC. Individuals homozygous for the A allele variant are predicted to have a decrease in the amount of C2 domain NEDD4L produced from expression of isoform I. The common frequency of this allele suggests that any functional effect will be subtle, as small effects are expected for common variants in common disease. Variant 13 of NEDD4L is therefore a likely candidate single nucleotide polymorphism for association with phenotypes relevant for hypertension and enveloped viral infections. The variant a given subject has can direct the type of therapy to provide. Homozygous individuals for the A variant at position 13 will benefit from sodium restriction and will best respond to drugs that reduce plasma volume, such as diuretics (for example, furosemide, bumetanide or ethacrynic acid), than to drugs that act primarily as vasodilators, such as beta blockers. Tables:

TABLE 3
SNP discovery in human NEDD4L exons and intron flanks.

| Coordinate* | | Coordinate** | Implication | Alleles | Frequency |
|---|---|---|---|---|---|
| variant 1 | 80950 | 825234 | 5′ flank | T/C | 0.16 |
| variant 2 | 80986 | 825270 | 5′ flank | C/G | 0.01 |
| variant 3 | 81117 | 825401 | 5′ flank | C/A | 0.47 |
| variant 4 | 81144 | 825428 | 5′ flank | G/A | 0.27 |
| variant 5 | 81189 | 825473 | 5′ flank | A/G | 0.22 |
| variant 6 | 81479 | 825765 | 5′flank | C/— | 0.47 |
| variant 7 | 81543 | 825828 | 5′ flank | C/T | 0.32 |
| variant 8 | 81756 | 826041 | 5′ flank | G/A | 0.49 |
| variant 9 | 82261 | 826546 | 5′ flank | G/A | 0.31 |
| variant 10 | 82369 | 826654 | 5′ flank | A/G | 0.21 |
| variant 11 | 82541 | 826826 | 5′ flank | G/A | 0.01 |
| variant 12 | 82578 | 826863 | 5′ flank | G/A | 0.01 |
| variant 13 | 82723 | 827008 | splice | G/A | 0.30 |
| | | | junction | | |
| variant 14 | 98767 | 843055 | intron 1 | A/G | 0.01 |
| variant 15 | 98830 | 843118 | intron 1 | C/T | 0.50 |
| variant 16 | 142069 | 871027 | intron 2 | G/A | 0.01 |
| variant 17 | 142182 | 871140 | intron 2 | C/T | 0.01 |
| variant 18 | 142210 | 871168 | intron 2 | A/C | 0.24 |
| variant 19 | 143720 | 872678 | exon2a | C/T | 0.01 |
| variant 20 | 143784 | 872742 | exon2a | G/A | 0.22 |
| variant 21 | 238195 | 925859 | intron 3 | C/G | 0.06 |
| variant 22 | 305555 | 993220 | intron 6 | T/C | 0.44 |
| variant 23 | 035589 | 993254 | intron 6 | A/G | 0.19 |
| variant 24 | 318797 | 1006462 | intron 10 | C/T | 0.38 |
| variant 25 | 320155 | 1007820 | intron 10 | TTCT/— | 0.22 |
| variant 26 | 330373 | 1018038 | intron 13 | G/A | 0.42 |
| variant 27 | 331039 | 1018704 | intron 14 | T/C | 0.01 |
| variant 28 | 331053 | 1018718 | intron 14 | GTT/— | 0.24 |
| variant 29 | 339121 | 1026786 | intron 17 | A/C | 0.01 |
| variant 30 | 359469 | 1047134 | intron 22 | G/A | 0.02 |
| variant 31 | 359494 | 1047159 | intron 22 | G/A | 0.22 |
| variant 32 | 359713 | 1047378 | intron 22 | T/C | 0.17 |
| variant 33 | 360074 | 1047739 | intron 23 | A/G | 0.21 |
| variant 34 | 362468 | 1050133 | intron 23 | TTAAA/— | 0.41 |
| variant 35 | 362874 | 1050539 | intron 24 | C/T | 0.04 |
| variant 36 | 375143 | 1062808 | intron 26 | G/C | 0.01 |
| variant 37 | 378727 | 1066392 | intron 28 | G/A | 0.01 |
| variant 38 | 386046 | 1073711 | 3′ UTR | T/C | 0.05 |

*Coordinates are from the human genome draft assembly hg8 06 Aug. 2001 freeze. The table coordinates can be indexed to chromosome 18 hg8 draft coordinates by adding 65,000,000 to each value.
**Coordinates are from SEQ ID NO: 1 and the human genomic sequence from NCBI, (“GenInfo Identifier”), contig NT_033907, gi = 27485382.

TABLE 3
Summary of NEDD4L genomic resequencing targets and exon
coordinates.

| Resequencing target* | | | Exon coordinates | | | |
|---|---|---|---|---|---|---|
| | | Length | | | | Length |
| Begin | End | (nt.) | | Begin | End | (nt.) |
| 80827 | 82835 | 2008 | exon1 | 82481 | 82723 | 242 |
| 98708 | 99315 | 607 | exon2 | 98954 | 99027 | 73 |
| 127963 | 128583 | 630 | exon2a | 143659 | 143902 | 243 |
| 234613 | 235239 | 626 | exon3 | 234885 | 234966 | 81 |
| 238062 | 238691 | 629 | exon4 | 238357 | 238395 | 38 |
| 241163 | 241797 | 634 | exon5 | 241459 | 241512 | 53 |
| 305150 | 305786 | 636 | exon6 | 305439 | 305489 | 50 |
| 311752 | 312995 | 1243 | exon7 | 311882 | 311943 | 61 |
| | | | exon8 | 312689 | 312791 | 102 |
| 314181 | 314805 | 624 | exon9 | 314453 | 314619 | 166 |
| 318155 | 318757 | 602 | exon10 | 318452 | 318584 | 132 |
| 319956 | 320592 | 636 | exon11 | 320195 | 320371 | 176 |
| 323037 | 323666 | 629 | exon12 | 323275 | 323349 | 74 |
| 324733 | 325219 | 486 | exon13 | 324935 | 324994 | 59 |
| 330241 | 331513 | 1272 | exon14 | 330495 | 330626 | 131 |
| | | | exon15 | 331135 | 331254 | 119 |
| 332149 | 332780 | 631 | exon16 | 332363 | 332560 | 197 |
| 338719 | 339341 | 622 | exon17 | 338994 | 339071 | 77 |
| 340213 | 340849 | 636 | exon18 | 340448 | 340502 | 54 |
| 346361 | 346992 | 631 | exon19 | 346651 | 346709 | 58 |
| 353435 | 354067 | 632 | exon20 | 353725 | 353790 | 65 |
| 355258 | 355899 | 641 | exon21 | 355456 | 355685 | 229 |
| 356971 | 357592 | 621 | exon22 | 357203 | 357324 | 121 |
| 359495 | 360059 | 564 | exon23 | 359854 | 359924 | 70 |
| 362367 | 362997 | 630 | exon24 | 362631 | 362726 | 95 |
| 372427 | 373048 | 621 | exon25 | 372703 | 372776 | 73 |
| 374717 | 375346 | 629 | exon26 | 374983 | 375043 | 60 |
| 376607 | 377237 | 630 | exon27 | 376896 | 376955 | 59 |
| 378280 | 378918 | 638 | exon28 | 378542 | 378649 | 107 |
| 379855 | 380491 | 636 | exon29 | 380103 | 380199 | 96 |
| 380678 | 381313 | 635 | exon30 | 380944 | 381016 | 72 |
| 385395 | 387844 | 2449 | exon31 | 385624 | 387615 | 1991 |
| Total: | 23108 nt. | | | | 5224 nt. | |

*Begin and end coordinates are from the human genome draft assembly hg8 Aug 06, 2001 freeze (www.genome.ucsc.edu, University of California, Santa Cruz). The table coordinates can be indexed to the chromosome 18 hg8 draft coordinates by adding 65,000,000 to each value. The resequencing target is the number of base pairs covered by high quality (phrap >= 20) sequence.

TABLE 4

Oligonucleotide sequences (SEQ ID NOS:2-119,
from left to right, top to bottom, respectively) for PCR
amplification and sequencing of human NEDD4L.

| | | |
|---|---|---|
| NEDD4L exon | PCR primers (5′-3′) | Sequencing primers (5′-3′) |

| | | |
|---|---|---|
| exon 1 | TCCGAGCAGAGCTCATGTAA | AGAGGCATCGAAGTACACGC |
| | GGCCAGCAAGAGAGATAGGA | AGGTTGCAGTGAGCCAAGAT |
| | | GCTGCCATAACAAAATACCCA |
| | | TCTAGCCCCTGGAATGTGAC |
| | | ATATTGGATTAGGGCCCACC |
| | | TATGAACCTGCCACCTGGAC |
| | | GAAAGTGGCAGAGAGGAAGC |
| | | AGATGAATTAACAGCTCCAGCA |
| exon 2 | GAGGAGAGTTCCAGCCACTG | TGGCAGGAGTAATTGATCTTAATG |
| | TGACTTCACGGTCAAGCAAG | AGAGCCATAGTCCCACACTG |
| exon 2a | GCCTGCTTTCTTCACTTCTGA | GGTGTTGTTTTGTTGCAAGG |
| | GACTGCACCACCCTGAAGA | AAATTTTCACTGTGCCGC |
| exon 3 | GGCTGAGGGTGTCCATAGAA | TGTAAAGTCTGAACAAAATCTGTGTG |
| | CATGGAGATAGGCAAATTAAGACA | GCTGAGGCAGGAGAATCACT |
| exon 4 | TGGAGGCTTTATTGAAGATGC | CTTGGTAGAATAATCATATCAATGCT |
| | GCTTTTCAGTCCTCCACAGG | CGTCTCATAGAATTAAACCATTCTCA |
| exon 5 | GAGCTGATAGATGCTGTTGGC | TCTACAGTCATGAGAATGCCTTG |
| | TGCACTGAATTATAGTGTCACTTCTG | GCCTACCGTTCTGTATTCCC |
| exon 6 | GGCAGCGTGATTAGGTCTGT | CAGCTGTGCCCTCAAACC |
| | AAGACCAGGTGCTGCTCACT | GCCTGAGTGAAGGAGGTGTC |
| exon 7, 8, 9 | AAACCCGGTGTGGATAGTGA | CGTGGACTGTCAGGTACCAA |
| | TCAGCTCATATTGAAAACAGCA | TCTAAAGAGAAATCTCTGCGAGG |
| | | GTGTCACCGACTGTGCAGAT |
| | | GTGACTCAAAGGCAATCGCT |
| exon 10, 11 | GTTCTTCAGACCCCGGCT | GCAAATGGGCAGAGGACTT |
| | TTGGATGAGCCACTTAAGCA | CCACCTATACTTGTCAAATACGGC |
| | | TGATTCACATCATGGCCCTA |
| | | CCAAGAAAGTGTTAGTGGTATAAGG |
| exon 12 | TCAAGGGGAAGAACATGAGC | TTCTGAAAGTTTTGGTCTCGTC |
| | TGTAAGGCAGGTGACGTGAG | TCAAAGTACAGATCACTGGAGCA |
| exon 13 | TGGAAACTGGTGATTCCTCC | TACCCAGGTCAAGGATGAGC |
| | ATGCAATTCCTGTGGCTTCT | TGCACTTAACCAAAGCCTGA |
| exon 14, 15, 16 | CCAAGCTGTGTTCACCTTCA | GGCAGCAACCCTTTACGTT |
| | ACTTTGTGAGACGGGATCAA | GGCAGGGAAGAAGCTACACA |
| | | TGGAAACTGGTGATTCCTCC |
| | | ATGCAATTCCTGTGGCTTCT |
| | | CTGAGTAGCTACCAAAGGATGG |
| | | GAGCTTCCCAGGAGAAAAGG |
| exon 17, 18 | AGTCAGCTCCTGGACTCTGG | CACTGCTGGGACTCTCTTCC |
| | ATGAAGAACATGGCACCTCC | GCCACTTAAATTGTCACCTGC |
| | | TTTTGAGCTAGCAGCCCATT |
| | | GCACTGTTCACAGATTTTTGACT |
| exon 19 | TGCACATTAACTCACTTGGAAGA | AAATCCTTAAAAGAACAGGGGTTT |
| | GATGGAAGAACCAATGGTGG | AGGAAGGGAGGGAATTTTGA |
| exon 20, 21 | TCAATTCCTGAGCCCTCAAC | CACCCTGACTCATATTCTGACAA |
| | AGCCTGGCAATGATGAAATC | TTGTGACTAGGGTTCTGAATTATTTT |
| | | CATCGCTAGTGGATGCCG |
| | | CCATGAACCCAATAGACAAGAA |
| exon 22, 23 | ATTTGGCTGAACCATATGAAA | CTTCGTAGGTCAAAAACAGTCAA |
| | CATGGTTCCAGGAAATGTGA | TTGCTTTCACTAAAGTTATTCCAGA |
| | | TCTGGCCTGCTACCCGC |
| | | AGATCAATGAACTTTTCAAGCAA |
| exon 24 | ACTGCTGGTTTGCCAGTCAT | TTTTGTAGACCAGTATGGCAGC |
| | GTGACACCTGCATCCACAG | AACCGAGGCACAAATGAAAC |
| exon 25, 26 | TTTCTGGAGTCTTGATGGGTG | GTTTCCTGATAAATGAATAGCAGAG |
| | GGGACTGCGCTTCATCAT | TCTTAGTTGATATCCACATATTCCAA |
| | | AAAGCATTTTAAAGCAGTTAAGCA |
| | | GTACCGCCTGAAACTCCAAG |
| exon 27, 28 | CACTGCCCAGATCGAACTTT | GCACTGACTCACAGCAGTGTT |
| | CACAGATCAAAATATTTGTTGGAA | CAGCTCTGCTCAGTGGCCT |
| | | CTAGAGGGGTAGGGAGGACG |
| | | GGAGGACCAGTCCCTCAAAT |
| exon 29, 30 | ATTATCTTAGGGCCACTGTTG | TGGTGCCATCAGAGAGTTTG |
| | TGACTTGGACACCATTTGGA | CGACCATATAAATGACCCTACAA |
| | | GCCCAGCTGAGAGGCTGT |
| | | CAGGTTGAGAGCTGCCTTATC |
| exon 31 | ATTGAGCCCTTGCTGTATGC | CTCCGAGATCTGCATCTGACT |
| | CAAGATGACAGAAGAATCCCAG | TTAATTCCATTCATTGTCATTAGAA |
| | | ACCTGGTCCCAGCTTGAGTT |
| | | TCGGATGAGAAATGGGAGTC |
| | | CCATACTCAGAATGAAAAACTGGA |
| | | TGAAGCATGCAACAGGTCAT |
| | | TCAATTGTGAATCTGGCTGC |
| | | GAAAAAGGAACCAACACTCAGC |
| | | AAGTTGTGGTTTGGGGAGGT |
| | | GGTTAGAATTGGATTTCTCCCTC |

TABLE 5

Oligonucleotide sequences (SEQ ID NOS:120-130,
respectively) for RT-PCR and RACE experiments.

| location | primer sequence |
|---|---|
| RT-PCR | |
| exon 1 | CCATTTCCAGAGAGGAACAACCGTG |
| isoform II | GCGCCGGCTCCATGGCGACC |
| exon 3 | AGCAAGTTCTCTATTCTCATCCGCT |
| RACE | |
| exon 2 | CTGGCTCCAAAGATGTCCTTTTTGG |
| | CGAGATCAATTCCAGAAACAAC |
| exon 2a | GAGTGGGAGGTGCCGTGCTGG |
| | GGAAGAGCTCGTCTGAAGTGG |
| exon 2c | GCTGCTGGAAATCTACCTTGG |
| | GGCTGGAAAGTGTTCAGCTGG |
| exon 3 | CCGCTACGTACAATGAAAGTTTCAC |
| | AGCAAGTTCTCTATTCTCATCCGCT |

TABLE 6
NEDD4L Association with Postural Blood Pressure Traits (Adjusted or
Age, Age, Age, and Sex) in Treated Hypertensives, Stratified by
Medication Class.

| | Medication Group 1* | | | Medication Group 2** | | |
|---|---|---|---|---|---|---|
| | Only | | | Only | | |
| Trait | AG or AA | GG | p-value | AG or AA | GG | p-value |
| N | 65 | 43 | | 71 | 57 | |
| delta SBP (t1) | −5.4 | −14.9 | 0.007 | 2.4 | 2.9 | 0.86*** |
| delta SBP (t2) | 3.6 | −1.4 | 0.08 | 5.3 | 8.8 | 0.22 |
| supine SBP | 135.2 | 137.8 | 0.47 | 130.2 | 124.3 | 0.07 |
| standing SBP | 129.8 | 122.9 | 0.10 | 132.5 | 127.2 | 0.17 |
| (t1) | | | | | | |
| standing SBP | 138.8 | 136.4 | 0.51 | 135.5 | 133.1 | 0.51 |
| (t2) | | | | | | |

*diuretics, calcium channel blockers, and/or adrenolytics
**beta blockers, ace inhibitors, and/or angiotensin II receptor antagonists
***p = 0.02 for formal test of genotype × medication group interaction

Tables 7-17 are indexed to SEQ ID NO: 1 and the genomic sequence from NCBI, (“GenInfo Identifier”), contig NT_{—}033907, gi=27485382.

TABLE 7
Isoform I exon-intron boundaries and translation start site.

| | | | | Translation |
|---|---|---|---|---|
| | | | | start |
| Genomic | Exon | Exon start | Exon end | coordinate |
| gi\|27485382:1-1080000 | 1_G | 826667 | 827008 | 826985 |
| gi\|27485382:1-1080000 | 2 | 843242 | 843315 | |
| gi\|27485382:1-1080000 | 3 | 922549 | 922630 | |
| gi\|27485382:1-1080000 | 4 | 926021 | 926059 | |
| gi\|27485382:1-1080000 | 5 | 929123 | 929176 | |
| gi\|27485382:1-1080000 | 6 | 993104 | 993154 | |
| gi\|27485382:1-1080000 | 7 | 999547 | 999608 | |
| gi\|27485382:1-1080000 | 8 | 1000354 | 1000456 | |
| gi\|27485382:1-1080000 | 9 | 1002118 | 1002284 | |
| gi\|27485382:1-1080000 | 10 | 1006117 | 1006249 | |
| gi\|27485382:1-1080000 | 11 | 1007860 | 1008036 | |
| gi\|27485382:1-1080000 | 12 | 1010940 | 1011014 | |
| gi\|27485382:1-1080000 | 13 | 1018160 | 1018291 | |
| gi\|27485382:1-1080000 | 14 | 1018800 | 1018919 | |
| gi\|27485382:1-1080000 | 15 | 1020028 | 1020225 | |
| gi\|27485382:1-1080000 | 16 | 1026659 | 1026736 | |
| gi\|27485382:1-1080000 | 17 | 1028113 | 1028167 | |
| gi\|27485382:1-1080000 | 18 | 1034316 | 1034374 | |
| gi\|27485382:1-1080000 | 19 | 1041390 | 1041455 | |
| gi\|27485382:1-1080000 | 20 | 1043121 | 1043350 | |
| gi\|27485382:1-1080000 | 21 | 1044868 | 1044989 | |
| gi\|27485382:1-1080000 | 22 | 1047519 | 1047589 | |
| gi\|27485382:1-1080000 | 23 | 1050296 | 1050391 | |
| gi\|27485382:1-1080000 | 24 | 1060368 | 1060441 | |
| gi\|27485382:1-1080000 | 25 | 1062648 | 1062708 | |
| gi\|27485382:1-1080000 | 26 | 1064561 | 1064620 | |
| gi\|27485382:1-1080000 | 27 | 1066207 | 1066314 | |
| gi\|27485382:1-1080000 | 28 | 1067768 | 1067864 | |
| gi\|27485382:1-1080000 | 29 | 1068609 | 1068681 | |
| gi\|27485382:1-1080000 | 30 | 1073289 | 1075279 | |

Full length cDNA shown in SEQ ID NO: 181
Encoded Protein is shown in SEQ ID NO: 182

TABLE 8
Isoform generated by variant 13-A

| | | | | Translation |
|---|---|---|---|---|
| | | | | start |
| Genomic | Exon | Exon start | Exon end | coordinate |
| gi\|27485382:1-1080000 | 1_A | 826667 | 827018 | 826985 |
| gi\|27485382:1-1080000 | 2 | 843242 | 843315 | |
| gi\|27485382:1-1080000 | 3 | 922549 | 922630 | |
| gi\|27485382:1-1080000 | 4 | 926021 | 926059 | |
| gi\|27485382:1-1080000 | 5 | 929123 | 929176 | |
| gi\|27485382:1-1080000 | 6 | 993104 | 993154 | |
| gi\|27485382:1-1080000 | 7 | 999547 | 999608 | |
| gi\|27485382:1-1080000 | 8 | 1000354 | 1000456 | |
| gi\|27485382:1-1080000 | 9 | 1002118 | 1002284 | |
| gi\|27485382:1-1080000 | 10 | 1006117 | 1006249 | |
| gi\|27485382:1-1080000 | 11 | 1007860 | 1008036 | |
| gi\|27485382:1-1080000 | 12 | 1010940 | 1011014 | |
| gi\|27485382:1-1080000 | 13 | 1018160 | 1018291 | |
| gi\|27485382:1-1080000 | 14 | 1018800 | 1018919 | |
| gi\|27485382:1-1080000 | 15 | 1020028 | 1020225 | |
| gi\|27485382:1-1080000 | 16 | 1026659 | 1026736 | |
| gi\|27485382:1-1080000 | 17 | 1028113 | 1028167 | |
| gi\|27485382:1-1080000 | 18 | 1034316 | 1034374 | |
| gi\|27485382:1-1080000 | 19 | 1041390 | 1041455 | |
| gi\|27485382:1-1080000 | 20 | 1043121 | 1043350 | |
| gi\|27485382:1-1080000 | 21 | 1044868 | 1044989 | |
| gi\|27485382:1-1080000 | 22 | 1047519 | 1047589 | |
| gi\|27485382:1-1080000 | 23 | 1050296 | 1050391 | |
| gi\|27485382:1-1080000 | 24 | 1060368 | 1060441 | |
| gi\|27485382:1-1080000 | 25 | 1062648 | 1062708 | |
| gi\|27485382:1-1080000 | 26 | 1064561 | 1064620 | |
| gi\|27485382:1-1080000 | 27 | 1066207 | 1066314 | |
| gi\|27485382:1-1080000 | 28 | 1067768 | 1067864 | |
| gi\|27485382:1-1080000 | 29 | 1068609 | 1068681 | |
| gi\|27485382:1-1080000 | 30 | 1073289 | 1075279 | |

Full length DNA = SEQ ID NO: 183
Protein = SEQ ID NO: 184

TABLE 9
Isoform generated by a novel exon 1 (Exon 1a)

| | | | | Translation |
|---|---|---|---|---|
| Genomic | Exon | Exon start | Exon end | start coordinate |
| gi\|27485382:1-1080000 | 1a | 722213 | 722534 | 722487 |
| gi\|27485382:1-1080000 | 2 | 843242 | 843315 | |
| gi\|27485382:1-1080000 | 3 | 922549 | 922630 | |
| gi\|27485382:1-1080000 | 4 | 926021 | 926059 | |
| gi\|27485382:1-1080000 | 5 | 929123 | 929176 | |
| gi\|27485382:1-1080000 | 6 | 993104 | 993154 | |
| gi\|27485382:1-1080000 | 7 | 999547 | 999608 | |
| gi\|27485382:1-1080000 | 8 | 1000354 | 1000456 | |
| gi\|27485382:1-1080000 | 9 | 1002118 | 1002284 | |
| gi\|27485382:1-1080000 | 10 | 1006117 | 1006249 | |
| gi\|27485382:1-1080000 | 11 | 1007860 | 1008036 | |
| gi\|27485382:1-1080000 | 12 | 1010940 | 1011014 | |
| gi\|27485382:1-1080000 | 13 | 1018160 | 1018291 | |
| gi\|27485382:1-1080000 | 14 | 1018800 | 1018919 | |
| gi\|27485382:1-1080000 | 15 | 1020028 | 1020225 | |
| gi\|27485382:1-1080000 | 16 | 1026659 | 1026736 | |
| gi\|27485382:1-1080000 | 17 | 1028113 | 1028167 | |
| gi\|27485382:1-1080000 | 18 | 1034316 | 1034374 | |
| gi\|27485382:1-1080000 | 19 | 1041390 | 1041455 | |
| gi\|27485382:1-1080000 | 20 | 1043121 | 1043350 | |
| gi\|27485382:1-1080000 | 21 | 1044868 | 1044989 | |
| gi\|27485382:1-1080000 | 22 | 1047519 | 1047589 | |
| gi\|27485382:1-1080000 | 23 | 1050296 | 1050391 | |
| gi\|27485382:1-1080000 | 24 | 1060368 | 1060441 | |
| gi\|27485382:1-1080000 | 25 | 1062648 | 1062708 | |
| gi\|27485382:1-1080000 | 26 | 1064561 | 1064620 | |
| gi\|27485382:1-1080000 | 27 | 1066207 | 1066314 | |
| gi\|27485382:1-1080000 | 28 | 1067768 | 1067864 | |
| gi\|27485382:1-1080000 | 29 | 1068609 | 1068681 | |
| gi\|27485382:1-1080000 | 30 | 1073289 | 1075279 | |

Full length DNA = SEQ ID NO: 185
Protein = SEQ ID NO: 186

TABLE 10
Isoform generated by novel exon 1 (Exon 1b)

| | | | | Translation |
|---|---|---|---|---|
| Genomic | Exon | Exon start | Exon end | start coordinate |
| gi\|27485382:1-1080000 | 1b | 723319 | 723442 | |
| gi\|27485382:1-1080000 | 2 | 843242 | 843315 | |
| gi\|27485382:1-1080000 | 3 | 922549 | 922630 | |
| gi\|27485382:1-1080000 | 4 | 926021 | 926059 | |
| gi\|27485382:1-1080000 | 5 | 929123 | 929176 | |
| gi\|27485382:1-1080000 | 6 | 993104 | 993154 | |
| gi\|27485382:1-1080000 | 7 | 999547 | 999608 | 999562 |
| gi\|27485382:1-1080000 | 8 | 1000354 | 1000456 | |
| gi\|27485382:1-1080000 | 9 | 1002118 | 1002284 | |
| gi\|27485382:1-1080000 | 10 | 1006117 | 1006249 | |
| gi\|27485382:1-1080000 | 11 | 1007860 | 1008036 | |
| gi\|27485382:1-1080000 | 12 | 1010940 | 1011014 | |
| gi\|27485382:1-1080000 | 13 | 1018160 | 1018291 | |
| gi\|27485382:1-1080000 | 14 | 1018800 | 1018919 | |
| gi\|27485382:1-1080000 | 15 | 1020028 | 1020225 | |
| gi\|27485382:1-1080000 | 16 | 1026659 | 1026736 | |
| gi\|27485382:1-1080000 | 17 | 1028113 | 1028167 | |
| gi\|27485382:1-1080000 | 18 | 1034316 | 1034374 | |
| gi\|27485382:1-1080000 | 19 | 1041390 | 1041455 | |
| gi\|27485382:1-1080000 | 20 | 1043121 | 1043350 | |
| gi\|27485382:1-1080000 | 21 | 1044868 | 1044989 | |
| gi\|27485382:1-1080000 | 22 | 1047519 | 1047589 | |
| gi\|27485382:1-1080000 | 23 | 1050296 | 1050391 | |
| gi\|27485382:1-1080000 | 24 | 1060368 | 1060441 | |
| gi\|27485382:1-1080000 | 25 | 1062648 | 1062708 | |
| gi\|27485382:1-1080000 | 26 | 1064561 | 1064620 | |
| gi\|27485382:1-1080000 | 27 | 1066207 | 1066314 | |
| gi\|27485382:1-1080000 | 28 | 1067768 | 1067864 | |
| gi\|27485382:1-1080000 | 29 | 1068609 | 1068681 | |
| gi\|27485382:1-1080000 | 30 | 1073289 | 1075279 | |

Full length DNA = SEQ ID NO: 187
Protein = SEQ ID NO: 188

TABLE 11
Isoform generated by novel exon 1 (Exon 1c)

| | | | | Translation |
|---|---|---|---|---|
| Genomic | Exon | Exon start | Exon end | start coordinate |
| gi\|27485382:1-1080000 | 1c | 723483 | 723516 | 723508 |
| gi\|27485382:1-1080000 | 2 | 843242 | 843315 | |
| gi\|27485382:1-1080000 | 3 | 922549 | 922630 | |
| gi\|27485382:1-1080000 | 4 | 926021 | 926059 | |
| gi\|27485382:1-1080000 | 5 | 929123 | 929176 | |
| gi\|27485382:1-1080000 | 6 | 993104 | 993154 | |
| gi\|27485382:1-1080000 | 7 | 999547 | 999608 | |
| gi\|27485382:1-1080000 | 8 | 1000354 | 1000456 | |
| gi\|27485382:1-1080000 | 9 | 1002118 | 1002284 | |
| gi\|27485382:1-1080000 | 10 | 1006117 | 1006249 | |
| gi\|27485382:1-1080000 | 11 | 1007860 | 1008036 | |
| gi\|27485382:1-1080000 | 12 | 1010940 | 1011014 | |
| gi\|27485382:1-1080000 | 13 | 1018160 | 1018291 | |
| gi\|27485382:1-1080000 | 14 | 1018800 | 1018919 | |
| gi\|27485382:1-1080000 | 15 | 1020028 | 1020225 | |
| gi\|27485382:1-1080000 | 16 | 1026659 | 1026736 | |
| gi\|27485382:1-1080000 | 17 | 1028113 | 1028167 | |
| gi\|27485382:1-1080000 | 18 | 1034316 | 1034374 | |
| gi\|27485382:1-1080000 | 19 | 1041390 | 1041455 | |
| gi\|27485382:1-1080000 | 20 | 1043121 | 1043350 | |
| gi\|27485382:1-1080000 | 21 | 1044868 | 1044989 | |
| gi\|27485382:1-1080000 | 22 | 1047519 | 1047589 | |
| gi\|27485382:1-1080000 | 23 | 1050296 | 1050391 | |
| gi\|27485382:1-1080000 | 24 | 1060368 | 1060441 | |
| gi\|27485382:1-1080000 | 25 | 1062648 | 1062708 | |
| gi\|27485382:1-1080000 | 26 | 1064561 | 1064620 | |
| gi\|27485382:1-1080000 | 27 | 1066207 | 1066314 | |
| gi\|27485382:1-1080000 | 28 | 1067768 | 1067864 | |
| gi\|27485382:1-1080000 | 29 | 1068609 | 1068681 | |
| gi\|27485382:1-1080000 | 30 | 1073289 | 1075279 | |

Full length DNA = SEQ ID NO: 189
Protein = SEQ ID NO: 190

TABLE 12
Isoform generated by novel exon 1 (Exon 1d)

| | | | | Translation |
|---|---|---|---|---|
| Genomic | Exon | Exon start | Exon end | start coordinate |
| gi\|27485382:1-1080000 | 1d | 723499 | 723614 | |
| gi\|27485382:1-1080000 | 2 | 843242 | 843315 | |
| gi\|27485382:1-1080000 | 3 | 922549 | 922630 | |
| gi\|27485382:1-1080000 | 4 | 926021 | 926059 | |
| gi\|27485382:1-1080000 | 5 | 929123 | 929176 | |
| gi\|27485382:1-1080000 | 6 | 993104 | 993154 | |
| gi\|27485382:1-1080000 | 7 | 999547 | 999608 | 999562 |
| gi\|27485382:1-1080000 | 8 | 1000354 | 1000456 | |
| gi\|27485382:1-1080000 | 9 | 1002118 | 1002284 | |
| gi\|27485382:1-1080000 | 10 | 1006117 | 1006249 | |
| gi\|27485382:1-1080000 | 11 | 1007860 | 1008036 | |
| gi\|27485382:1-1080000 | 12 | 1010940 | 1011014 | |
| gi\|27485382:1-1080000 | 13 | 1018160 | 1018291 | |
| gi\|27485382:1-1080000 | 14 | 1018800 | 1018919 | |
| gi\|27485382:1-1080000 | 15 | 1020028 | 1020225 | |
| gi\|27485382:1-1080000 | 16 | 1026659 | 1026736 | |
| gi\|27485382:1-1080000 | 17 | 1028113 | 1028167 | |
| gi\|27485382:1-1080000 | 18 | 1034316 | 1034374 | |
| gi\|27485382:1-1080000 | 19 | 1041390 | 1041455 | |
| gi\|27485382:1-1080000 | 20 | 1043121 | 1043350 | |
| gi\|27485382:1-1080000 | 21 | 1044868 | 1044989 | |
| gi\|27485382:1-1080000 | 22 | 1047519 | 1047589 | |
| gi\|27485382:1-1080000 | 23 | 1050296 | 1050391 | |
| gi\|27485382:1-1080000 | 24 | 1060368 | 1060441 | |
| gi\|27485382:1-1080000 | 25 | 1062648 | 1062708 | |
| gi\|27485382:1-1080000 | 26 | 1064561 | 1064620 | |
| gi\|27485382:1-1080000 | 27 | 1066207 | 1066314 | |
| gi\|27485382:1-1080000 | 28 | 1067768 | 1067864 | |
| gi\|27485382:1-1080000 | 29 | 1068609 | 1068681 | |
| gi\|27485382:1-1080000 | 30 | 1073289 | 1075279 | |

Full length DNA = SEQ ID NO: 191
Protein = SEQ ID NO: 192

TABLE 13
Isoform generated by a novel exon (Exon 1e)

| | | | | Translation |
|---|---|---|---|---|
| Genomic | Exon | Exon start | Exon end | start coordinate |
| gi\|27485382:1-1080000 | 1e | 723738 | 723844 | |
| gi\|27485382:1-1080000 | 2 | 843242 | 843315 | |
| gi\|27485382:1-1080000 | 3 | 922549 | 922630 | |
| gi\|27485382:1-1080000 | 4 | 926021 | 926059 | |
| gi\|27485382:1-1080000 | 5 | 929123 | 929176 | |
| gi\|27485382:1-1080000 | 6 | 993104 | 993154 | |
| gi\|27485382:1-1080000 | 7 | 999547 | 999608 | 999562 |
| gi\|27485382:1-1080000 | 8 | 1000354 | 1000456 | |
| gi\|27485382:1-1080000 | 9 | 1002118 | 1002284 | |
| gi\|27485382:1-1080000 | 10 | 1006117 | 1006249 | |
| gi\|27485382:1-1080000 | 11 | 1007860 | 1008036 | |
| gi\|27485382:1-1080000 | 12 | 1010940 | 1011014 | |
| gi\|27485382:1-1080000 | 13 | 1018160 | 1018291 | |
| gi\|27485382:1-1080000 | 14 | 1018800 | 1018919 | |
| gi\|27485382:1-1080000 | 15 | 1020028 | 1020225 | |
| gi\|27485382:1-1080000 | 16 | 1026659 | 1026736 | |
| gi\|27485382:1-1080000 | 17 | 1028113 | 1028167 | |
| gi\|27485382:1-1080000 | 18 | 1034316 | 1034374 | |
| gi\|27485382:1-1080000 | 19 | 1041390 | 1041455 | |
| gi\|27485382:1-1080000 | 20 | 1043121 | 1043350 | |
| gi\|27485382:1-1080000 | 21 | 1044868 | 1044989 | |
| gi\|27485382:1-1080000 | 22 | 1047519 | 1047589 | |
| gi\|27485382:1-1080000 | 23 | 1050296 | 1050391 | |
| gi\|27485382:1-1080000 | 24 | 1060368 | 1060441 | |
| gi\|27485382:1-1080000 | 25 | 1062648 | 1062708 | |
| gi\|27485382:1-1080000 | 26 | 1064561 | 1064620 | |
| gi\|27485382:1-1080000 | 27 | 1066207 | 1066314 | |
| gi\|27485382:1-1080000 | 28 | 1067768 | 1067864 | |
| gi\|27485382:1-1080000 | 29 | 1068609 | 1068681 | |
| gi\|27485382:1-1080000 | 30 | 1073289 | 1075279 | |

Full length DNA = SEQ ID NO: 193
Protein = SEQ ID NO: 194

TABLE 14
Isoform III (Exon 2a-exon3)

| | | | | Translation |
|---|---|---|---|---|
| Genomic | Exon | Exon start | Exon end | start coordinate |
| gi\|27485382:1-1080000 | 2a | 872617 | 872860 | |
| gi\|27485382:1-1080000 | 3 | 922549 | 922630 | |
| gi\|27485382:1-1080000 | 4 | 926021 | 926059 | |
| gi\|27485382:1-1080000 | 5 | 929123 | 929176 | |
| gi\|27485382:1-1080000 | 6 | 993104 | 993154 | |
| gi\|27485382:1-1080000 | 7 | 999547 | 999608 | 999562 |
| gi\|27485382:1-1080000 | 8 | 1000354 | 1000456 | |
| gi\|27485382:1-1080000 | 9 | 1002118 | 1002284 | |
| gi\|27485382:1-1080000 | 10 | 1006117 | 1006249 | |
| gi\|27485382:1-1080000 | 11 | 1007860 | 1008036 | |
| gi\|27485382:1-1080000 | 12 | 1010940 | 1011014 | |
| gi\|27485382:1-1080000 | 13 | 1018160 | 1018291 | |
| gi\|27485382:1-1080000 | 14 | 1018800 | 1018919 | |
| gi\|27485382:1-1080000 | 15 | 1020028 | 1020225 | |
| gi\|27485382:1-1080000 | 16 | 1026659 | 1026736 | |
| gi\|27485382:1-1080000 | 17 | 1028113 | 1028167 | |
| gi\|27485382:1-1080000 | 18 | 1034316 | 1034374 | |
| gi\|27485382:1-1080000 | 19 | 1041390 | 1041455 | |
| gi\|27485382:1-1080000 | 20 | 1043121 | 1043350 | |
| gi\|27485382:1-1080000 | 21 | 1044868 | 1044989 | |
| gi\|27485382:1-1080000 | 22 | 1047519 | 1047589 | |
| gi\|27485382:1-1080000 | 23 | 1050296 | 1050391 | |
| gi\|27485382:1-1080000 | 24 | 1060368 | 1060441 | |
| gi\|27485382:1-1080000 | 25 | 1062648 | 1062708 | |
| gi\|27485382:1-1080000 | 26 | 1064561 | 1064620 | |
| gi\|27485382:1-1080000 | 27 | 1066207 | 1066314 | |
| gi\|27485382:1-1080000 | 28 | 1067768 | 1067864 | |
| gi\|27485382:1-1080000 | 29 | 1068609 | 1068681 | |
| gi\|27485382:1-1080000 | 30 | 1073289 | 1075279 | |

Full length DNA = SEQ ID NO: 195
Protein = SEQ ID NO: 196

TABLE 15
Isoform VI (Exon 2b-Exon 3)

| | | | | Translation |
|---|---|---|---|---|
| Genomic | Exon | Exon start | Exon end | start coordinate |
| gi\|27485382:1-1080000 | 2b | 874858 | 874996 | |
| gi\|27485382:1-1080000 | 3 | 922549 | 922630 | |
| gi\|27485382:1-1080000 | 4 | 926021 | 926059 | |
| gi\|27485382:1-1080000 | 5 | 929123 | 929176 | |
| gi\|27485382:1-1080000 | 6 | 993104 | 993154 | 8 |
| gi\|27485382:1-1080000 | 7 | 999547 | 999608 | 999562 |
| gi\|27485382:1-1080000 | 8 | 1000354 | 1000456 | |
| gi\|27485382:1-1080000 | 9 | 1002118 | 1002284 | |
| gi\|27485382:1-1080000 | 10 | 1006117 | 1006249 | |
| gi\|27485382:1-1080000 | 11 | 1007860 | 1008036 | |
| gi\|27485382:1-1080000 | 12 | 1010940 | 1011014 | |
| gi\|27485382:1-1080000 | 13 | 1018160 | 1018291 | |
| gi\|27485382:1-1080000 | 14 | 1018800 | 1018919 | |
| gi\|27485382:1-1080000 | 15 | 1020028 | 1020225 | |
| gi\|27485382:1-1080000 | 16 | 1026659 | 1026736 | |
| gi\|27485382:1-1080000 | 17 | 1028113 | 1028167 | |
| gi\|27485382:1-1080000 | 18 | 1034316 | 1034374 | |
| gi\|27485382:1-1080000 | 19 | 1041390 | 1041455 | |
| gi\|27485382:1-1080000 | 20 | 1043121 | 1043350 | |
| gi\|27485382:1-1080000 | 21 | 1044868 | 1044989 | |
| gi\|27485382:1-1080000 | 22 | 1047519 | 1047589 | |
| gi\|27485382:1-1080000 | 23 | 1050296 | 1050391 | |
| gi\|27485382:1-1080000 | 24 | 1060368 | 1060441 | |
| gi\|27485382:1-1080000 | 25 | 1062648 | 1062708 | |
| gi\|27485382:1-1080000 | 26 | 1064561 | 1064620 | |
| gi\|27485382:1-1080000 | 27 | 1066207 | 1066314 | |
| gi\|27485382:1-1080000 | 28 | 1067768 | 1067864 | |
| gi\|27485382:1-1080000 | 29 | 1068609 | 1068681 | |
| gi\|27485382:1-1080000 | 30 | 1073289 | 1075279 | |

Full length DNA = SEQ ID NO: 197
Protein = SEQ ID NO: 198

TABLE 16
Isoform IV (Exon 2c-Exon 3)

| | | | | Translation |
|---|---|---|---|---|
| Genomic | Exon | Exon start | Exon end | start coordinate |
| gi\|27485382:1-1080000 | 2c | 898644 | 898879 | |
| gi\|27485382:1-1080000 | 3 | 922549 | 922630 | |
| gi\|27485382:1-1080000 | 4 | 926021 | 926059 | |
| gi\|27485382:1-1080000 | 5 | 929123 | 929176 | |
| gi\|27485382:1-1080000 | 6 | 993104 | 993154 | |
| gi\|27485382:1-1080000 | 7 | 999547 | 999608 | 999562 |
| gi\|27485382:1-1080000 | 8 | 1000354 | 1000456 | |
| gi\|27485382:1-1080000 | 9 | 1002118 | 1002284 | |
| gi\|27485382:1-1080000 | 10 | 1006117 | 1006249 | |
| gi\|27485382:1-1080000 | 11 | 1007860 | 1008036 | |
| gi\|27485382:1-1080000 | 12 | 1010940 | 1011014 | |
| gi\|27485382:1-1080000 | 13 | 1018160 | 1018291 | |
| gi\|27485382:1-1080000 | 14 | 1018800 | 1018919 | |
| gi\|27485382:1-1080000 | 15 | 1020028 | 1020225 | |
| gi\|27485382:1-1080000 | 16 | 1026659 | 1026736 | |
| gi\|27485382:1-1080000 | 17 | 1028113 | 1028167 | |
| gi\|27485382:1-1080000 | 18 | 1034316 | 1034374 | |
| gi\|27485382:1-1080000 | 19 | 1041390 | 1041455 | |
| gi\|27485382:1-1080000 | 20 | 1043121 | 1043350 | |
| gi\|27485382:1-1080000 | 21 | 1044868 | 1044989 | |
| gi\|27485382:1-1080000 | 22 | 1047519 | 1047589 | |
| gi\|27485382:1-1080000 | 23 | 1050296 | 1050391 | |
| gi\|27485382:1-1080000 | 24 | 1060368 | 1060441 | |
| gi\|27485382:1-1080000 | 25 | 1062648 | 1062708 | |
| gi\|27485382:1-1080000 | 26 | 1064561 | 1064620 | |
| gi\|27485382:1-1080000 | 27 | 1066207 | 1066314 | |
| gi\|27485382:1-1080000 | 28 | 1067768 | 1067864 | |
| gi\|27485382:1-1080000 | 29 | 1068609 | 1068681 | |
| gi\|27485382:1-1080000 | 30 | 1073289 | 1075279 | |

Full length DNA = SEQ ID NO: 199
Protein = SEQ ID NO: 200

TABLE 17
Isoform V (Exon 2a-Exon 2c-Exon 3)

| | | | | Translation |
|---|---|---|---|---|
| Genomic | Exon | Exon start | Exon end | start coordinate |
| gi\|27485382:1-1080000 | 2a | 872617 | 872860 | |
| gi\|27485382:1-1080000 | 2d | 904952 | 905030 | |
| gi\|27485382:1-1080000 | 3 | 922549 | 922631 | |
| gi\|27485382:1-1080000 | 4 | 926021 | 926060 | |
| gi\|27485382:1-1080000 | 5 | 929123 | 929177 | |
| gi\|27485382:1-1080000 | 6 | 993104 | 993155 | |
| gi\|27485382:1-1080000 | 7 | 999547 | 999609 | 999562 |
| gi\|27485382:1-1080000 | 8 | 1000354 | 1000457 | |
| gi\|27485382:1-1080000 | 9 | 1002118 | 1002285 | |
| gi\|27485382:1-1080000 | 10 | 1006117 | 1006250 | |
| gi\|27485382:1-1080000 | 11 | 1007860 | 1008037 | |
| gi\|27485382:1-1080000 | 12 | 1010940 | 1011015 | |
| gi\|27485382:1-1080000 | 13 | 1018160 | 1018292 | |
| gi\|27485382:1-1080000 | 14 | 1018800 | 1018920 | |
| gi\|27485382:1-1080000 | 15 | 1020028 | 1020226 | |
| gi\|27485382:1-1080000 | 16 | 1026659 | 1026737 | |
| gi\|27485382:1-1080000 | 17 | 1028113 | 1028168 | |
| gi\|27485382:1-1080000 | 18 | 1034316 | 1034375 | |
| gi\|27485382:1-1080000 | 19 | 1041390 | 1041456 | |
| gi\|27485382:1-1080000 | 20 | 1043121 | 1043351 | |
| gi\|27485382:1-1080000 | 21 | 1044868 | 1044990 | |
| gi\|27485382:1-1080000 | 22 | 1047519 | 1047590 | |
| gi\|27485382:1-1080000 | 23 | 1050296 | 1050392 | |
| gi\|27485382:1-1080000 | 24 | 1060368 | 1060442 | |
| gi\|27485382:1-1080000 | 25 | 1062648 | 1062709 | |
| gi\|27485382:1-1080000 | 26 | 1064561 | 1064621 | |
| gi\|27485382:1-1080000 | 27 | 1066207 | 1066315 | |
| gi\|27485382:1-1080000 | 28 | 1067768 | 1067865 | |
| gi\|27485382:1-1080000 | 29 | 1068609 | 1068682 | |
| gi\|27485382:1-1080000 | 30 | 1073289 | 1075280 | |

Full length DNA = SEQ ID NO: 201
Protein = SEQ ID NO: 202

### REFERENCES

1. Corvol P, Persu A, Gimenez-Roqueplo A P and Jeunemaitre X. Seven lessons from two candidate genes in human essential hypertension: angiotensinogen and epithelial sodium channel. Hypertension 33: 1324-1331, 1999.
2. Luft F C. Molecular genetics of human hypertension. Curr Opin Nephrol Hypertens 9: 259-266, 2000.
3. Lifton R P, Gharavi A G and Geller D S. Molecular mechanisms of human hypertension. Cell 104: 545-556, 2001.
4. Liddle G W, Bledsoe T and Coppage W S, Jr. A familial renal disorder simulating primary aldosteronism but with negligible aldosterone secretion. Trans. Assoc. Am. Physicians 76: 199-213, 1963.
5. Shimkets R A, Warnock D G, Bositis C M, Nelson-Williams C, Hansson J H, Schambelan M, Gill J R, Jr., Ulick S, Milora R V, Findling J W and et al. Liddle's syndrome: heritable human hypertension caused by mutations in the beta subunit of the epithelial sodium channel. Cell 79: 407-414, 1994.
6. Schild L, Canessa C M, Shimkets R A, Gautschi I, Lifton R P and Rossier B C. A mutation in the epithelial sodium channel causing Liddle disease increases channel activity in the Xenopus laevis oocyte expression system. Proc Natl Acad Sci USA 92: 5699-5703, 1995.
7. Debonneville C, Flores S Y, Kamynina E, Plant P J, Tauxe C, Thomas M A, Munster C, Chraibi A, Pratt J H, Horisberger J D, Pearce D, Loffing J and Staub O. Phosphorylation of Nedd4-2 by Sgkl regulates epithelial Na(+) channel cell surface expression. Embo J20: 7052-7059, 2001.
8. Snyder P M, Olson D R and Thomas B C. Serum and Glucocorticoid-regulated Kinase Modulates Nedd4-2-mediated Inhibition of the Epithelial Na+ Channel. J Bio Chem 277: 5-8, 2002.
9. Lemmon S K and Traub L M. Sorting in the endosomal system in yeast and animal cells. Curr Opin Cell Bio 12: 457-466, 2000.
10. Staub O, Dho S, Henry P, Correa J, Ishikawa T, McGlade J and Rotin D. W W domains of Nedd4 bind to the proline-rich PY motifs in the epithelial Na+ channel deleted in Liddle's syndrome. Embo J 15: 2371-2380, 1996.
11. Kamynina E, Tauxe C and Staub O. Distinct characteristics of two human Nedd4 proteins with respect to epithelial Na(+) channel regulation. Am J Physiol Renal Physiol 281: F469-477, 2001a.
12. Kamynina E, Debonneville C, Bens M, Vandewalle A and Staub O. A novel mouse Nedd4 protein suppresses the activity of the epithelial Na+ channel. Faseb J 15: 204-214, 2001b.
13. Harvey K F, Dinudom A, Cook D I and Kumar S. The Nedd4-like protein KIAA0439 (SEQ ID NO: 154) is a potential regulator of the epithelial sodium channel. J Bio Chem 276: 8597-8601, 2001.
14. Atwood L D, Samollow P B, Hixson J E, Stern M P and MacCluer J W. Genome-wide linkage analysis of blood pressure in Mexican Americans. Genet Epidemiol 20: 373-382, 2001.
15. Pankow J S, Rose K M, Oberman A, Hunt S C, Atwood L D, Djousse L, Province M A and Rao D C. Possible locus on chromosome 18q influencing postural systolic blood pressure changes. Hypertension 36: 471-476, 2000.
16. DeStefano A L, Baldwin C T, Burzstyn M, Gavras I, Handy D E, Joost O, Martel T, Nicolaou M, Schwartz F, Streeten D H, Farrer L A and Gavras H. Autosomal dominant orthostatic hypotensive disorder maps to chromosome 18q. Am J Hum Genet 63: 1425-1430, 1998.
17. Williams R R, Rao D C, Ellison R C, Arnett D K, Heiss G, Oberman A, Eckfeldt J H, Leppert M F, Province M A, Mockrin S C and Hunt S C. NHLBI family blood pressure program: methodology and recruitment in the HyperGEN network. Hypertension genetic epidemiology network. Ann Epidemioll O: 389-400, 2000.
18. Ewing B, Hillier L, Wendl M C and Green P. Base-calling of automated sequencer traces using phred. I. Accuracy assessment. Genome Res 8: 175-185, 1998.
19. Nickerson D A, Tobe V O and Taylor S L. PolyPhred: automating the detection and genotyping of single nucleotide substitutions using fluorescence-based resequencing. Nucleic Acids Res 25: 2745-2751, 1997.
20. Stephens R M and Schneider T D. Features of spliceosome evolution and function inferred from an analysis of the information at human splice sites. J Mol Bioi 228: 1124-1136, 1992.
21. Nakai K and Sakamoto H. Construction of a novel database containing aberrant splicing mutations of mammalian genes. Gene 141: 171-177, 1994.
22. Rogan P K, Faux B M and Schneider T D. Information analysis of human splice site mutations. Hum Mutat 12: 153-171, 1998.
23. Schultz J, Copley R R, Doerks T, Ponting C P and Bork P. SMART: a web-based tool for the study of genetically mobile domains. Nucleic Acids Research 28: 231-234, 2000.
24. Rebhun J F and Pratt J H. Molecular cloning of Nedd4 from Xenopus laevis. DNA Seq 9: 295-306, 1998.
25. Makalowski W, Zhang J and Boguski M S. Comparative analysis of 1196 orthologous mouse and human full-length mRNA and protein sequences. Genome Res 6: 846-857, 1996.
26. Plant P J, Yeger H, Staub O, Howard P and Rotin D. The C2 domain of the ubiquitin protein ligase Nedd4 mediates Ca2+-dependent plasma membrane localization. J Bioi Chem 272: 32329-32336, 1997.
27. Plant P J, Lafont F, Lecat S, Verkade P, Simons K and Rotin D. Apical membrane targeting of Nedd4 is mediated by an association of its C2 domain with annex in XIIlb. J Cell Bioi 149: 1473-1484, 2000.
28. Ikonen E. Roles of lipid rafts in membrane transport. Curr Opin Cell Bioi 13: 470-477, 2001.
29. Snyder P M, Olson D R, McDonald F J and Bucher D B. Multiple WW domains, but not the C2 domain, are required for inhibition of the epithelial Na+channel by human Nedd4. J Bioi Chem 276: 28321-28326, 2001.
30. Dunn Rand Hicke L. Domains of the Rsp5 ubiquitin-protein ligase required for receptor-mediated and fluid-phase endocytosis. Mol Bioi Cell 12: 421-435, 2001.
31. Vogt V M. Ubiquitin in retrovirus assembly: actor or bystander? Proc Natl Acad Sci USA 97.: 12945-12947, 2000.
32. Hicke L. A new ticket for entry into budding vesicles-ubiquitin. Cell 106: 527-530, 2001.
33. Kikonyogo A, Bouamr F, Van a M L, Xiang Y, Aiyar A, Carter C and Leis J. Proteins related to the Nedd4 family of ubi quit in protein ligases interact with the L domain of Rous sarcoma virus and are required for gag budding from cells. Proc Natl Acad Sci USA 98: 11199-11204, 2001.
34. Strack B, Calistri A, Accola M A, Palu G and Gottlinger H G. A role for ubiquitin ligase recruitment in retrovirus release. In: Proceedings of the National Academy of Sciences PNAS, 2000, p. 13063-13068.

1. An isolated nucleic acid sequence comprising a nucleic acid sequence encoding a NEDD4L gene product having a Ca²⁺-dependent lipid binding (C2) domain, or a functional fragment thereof, wherein said functional fragment thereof comprises the Ca²⁺-dependent lipid binding (C2) domain.

2. The isolated nucleic acid sequence of claim 1, wherein the nucleic acid sequence encodes a NEDD4L gene product selected from the group consisting of SEQ ID NO: 188, SEQ ID NO: 190, SEQ ID NO: 192, SEQ ID NO: 194, SEQ ID NO: 198, SEQ ID NO:202, and a sequence having 95% identity thereto.

3. The isolated nucleic acid sequence of claim 1, wherein the nucleic acid encoding the NEDD4L gene product encodes a protein selected from the group consisting of SEQ ID NO: 182, SEQ ID NO: 186 and SEQ ID NO: 190.

4. The isolated nucleic acid sequence of claim 1, wherein the isolated nucleic acid is a cDNA or a PCR product.

5. An expression vector comprising the nucleic acid sequence of claim 1.

6. A host cell comprising the nucleic acid sequence of claim 1.

7. The host cell of claim 6, wherein said host cell is selected from the group consisting of E. Coli, Bacillus sp., Streptomyces sp., yeast, fungi, insect cells, plant cells and mammalian cells.

8. An isolated nucleic acid sequence comprising a sequence having at least one variant selected from the group consisting of variant 1, variant 2, variant 3, variant 4, variant 5, variant 6, variant 7, variant 8, variant 9, variant 10, variant 11, variant 12, variant 13, variant 14, variant 15, variant 16, variant 17, variant 18, variant 19, variant 20, variant 21, variant 22, variant 23, variant 24, variant 25, variant 26, variant 27, variant 28, variant 29, variant 30, variant 31, variant 32, variant 33, variant 34, variant 35, variant 36, variant 37, variant 38 and a GT microsatellite polymorphism linked to NEDD4L, wherein the sequence is useful in the diagnosis of hypertension.

9. The isolated nucleic acid sequence of claim 7, wherein the isolated nucleic acid is a cDNA or a PCR product.

10. The isolated nucleic acid sequence of claim 9, wherein the variant is variant 13.

11. The isolated nucleic acid sequence of claim 10, wherein the nucleic acid is a PCR product less than or equal to 1000 nucleotides in length.

12. The isolated nucleic acid sequence of claim 7, wherein said isolated nucleic acid is a cDNA.

13. An isolated polypeptide comprising NEDD4L having a Ca²⁺-dependent lipid binding (C2) domain.

14. The isolated polypeptide of claim 13, wherein NEDD4L is selected from the group consisting of SEQ ID NO: 182, SEQ ID NO: 186, SEQ ID NO: 190 and a sequence with at least 87% identity, or a functional fragment thereof, wherein said functional fragment comprises the Ca²⁺-dependent lipid binding (C2) domain.

15. The isolated polypeptide of claim 13, wherein NEDD4L is selected from the group consisting of SEQ ID NO:188, SEQ ID NO:192, SEQ ID NO:194, SEQ ID NO:198, SEQ ID NO:202, and a sequence having at least 87% identity thereto.

16. The isolated polypeptide of claim 14, wherein the sequence with at least 87% identity has at least 95% identity.

17. The isolated polypeptide of claim 15, wherein the sequence with at least 87% identity has at least 95% identity.

18. An antibody or antibody fragment specifically recognizing the polypeptide of claim 13.

19. The antibody or antibody fragment of claim 18, wherein the antibody recognizes an epitope comprising the Ca²⁺-dependent lipid binding (C2) domain of NEDD4L.

20. A method of treating a subject thought to be in need of treatment of enveloped viral infection, hypertension or hypotension, said method comprising:
administering a therapeutic agent capable of reducing an amount of one or more isoforms of NEDD4L protein to the subject.

21. The method according to claim 20, comprising providing a nucleic acid encoding NEDD4L operably linked to a promoter; and expressing the nucleic acid in the subject.

22. The method according to claim 21, comprising administering to the subject a nucleic acid encoding NEDD4L having a Ca²⁺-dependent lipid binding (C2) domain.

23. The method according to claim 20, wherein the therapeutic agent comprises an antibody or antibody fragment.

24. The method according to claim 23, wherein the antibody or antibody fragment recognizes an epitope in a Ca²⁺-dependent lipid binding (C2) domain of NEDD4L.

25. The method according to claim 20, wherein the therapeutic agent comprises an antisence nucleic acid sequence.

26. A nucleic acid sequence for identifying sequence information about a NEDD4L gene, comprising a primer capable of providing sequence information about at least one variant selected from the group consisting of variant 1, variant 2, variant 3, variant 4, variant 5, variant 6, variant 7, variant 8, variant 9, variant 10, variant 11, variant 12, variant 13, variant 14, variant 15, variant 16, variant 17, variant 18, variant 19, variant 20, variant 21, variant 22, variant 23, variant 24, variant 25, variant 26, variant 27, variant 28, variant 29, variant 30, variant 31, variant 32, variant 33, variant 34, variant 35, variant 36, variant 37, variant 38 and a GT microsatellite polymorphism linked to NEDD4L.

27. The nucleic acid sequence of claim 26, wherein the at least one variant comprises variant 13, wherein position variant 13 is nucleotide position 82,773 of chromosome 18 of hg8 as disclosed in a Aug. 6, 2001 freeze with coordinates indexed to base 65,000,000.

28. The nucleic acid sequence of claim 26, wherein the nucleic acid sequence is a PCR primer.

29. The nucleic acid sequence of claim 26, wherein the nucleic acid sequence is an allele specific probe.

30. The nucleic acid of claim 26, wherein the GT microsatellite polymorphism linked to NEDD4L provides information about a polymorphism showing linkage with the variant 13.

31. The nucleic acid of claim 30, wherein the polymorphism is selected from the group consisting of a single nucleotide polymorphism, a restriction fragment polymorphism, a dinucleotide polymorphism, a trinucleotide polymorphism, a deletion and an insertion.

32. A method for diagnosing, prognosing or treating hypertension or enveloped viral infections in a subject, the method comprising:
obtaining a sample from a subject;
analyzing in the sample from the subject whether the subject is capable of producing a NEDD4L gene product having a Ca²⁺-dependent lipid binding C2 domain; and
diagnosing, prognosing or treating hypertension or an enveloped viral infection in the subject based on the capability of the subject to produce a NEDD4L gene product having the Ca²⁺-dependent lipid binding C2 domain.

33. A method according to claim 32, wherein analyzing the sample from the subject comprises assaying for the presence or absence of a NEDD4L protein having a Ca²⁺-dependent lipid binding C2 domain.

34. The method according to claim 33, wherein the NEDD4L protein is detected by immunoblotting, immunocytochemistry, enzyme-linked immunosorbent assay or affinity chromatography.

35. The method according to claim 32, wherein analyzing the sample from the subject comprises introducing a probe into the sample under conditions suitable for hybridization of the probe to a gene or mRNA sequence present in the sample, hybridizing the probe, and assaying hybridization of the probe.

36. The method according to claim 35, further comprising isolating genomic DNA or cDNA nucleic acid sequence from the sample.

37. The method according to claim 35, comprising hybridizing the probe to a mRNA sequence present in the sample.

38. The method according to claim 35, comprising providing an allele specific probe.

39. The method according to claim 35, comprising analyzing position variant 13.

40. The method according to claim 32, comprising determining an absence of NEDD4L protein having a Ca²⁺-dependent lipid binding C2 domain, wherein the absence is indicative of a vaso constricted condition.

41. The method according to claim 32, comprising diagnosing, prognosing or treating hypertension and selecting an appropriate antihypertension medication.

42. The method according to claim 41, wherein selecting an appropriate antihypertension medication comprises selecting a plasma volume reducing agent.

43. The method according to claim 42, wherein the plasma volume reducing agent is a diuretic.

44. The method according to claim 42, wherein the diuretic is selected from the group consisting of furosemide, bumetanide and ethacrynic acid.

45. The method according to claim 41, wherein selecting an appropriate antihypertension medication comprises selecting a drug having vasodilator activity.

46. A method of detecting interaction between NEDD4L and another molecule, the method comprising:
assaying for a binding interaction between a NEDD4L protein having a Ca²⁺-dependent lipid binding C2 domain and a binding partner capable of specifically binding the NEDD4L protein.

47. The method according to claim, comprising assaying for a binding interaction between a NEDD4L protein selected from the group consisting of SEQ ID NO: 182, SEQ ID NO: 186 and SEQ ID NO:190.

48. The method according to claim 46, further comprising determining a cellular localization of said binding partner.

49. The method according to claim 46, wherein the binding partner is a lipid.

50. A kit for identifying information about a NEDD4L gene or gene product, comprising:
at least one a probe or antibody, or antibody fragment, capable of determining the presence or absence of a NEDD4L Ca²⁺-dependent lipid binding C2 domain.

51. The kit of claim 50, wherein the probe provides information about a position variant selected from the group consisting of variant 1, variant 2, variant 3, variant 4, variant 5, variant 6, variant 7, variant 8, variant 9, variant 10, variant 11, variant 12, variant 14, variant 15, variant 16, variant 17, variant 18, variant 19, variant 20, variant 21, variant 22, variant 23, variant 24, variant 25, variant 26, variant 27, variant 28, variant 29, variant 30, variant 31, variant 32, variant 33, variant 34, variant 35, variant 36, variant 37, variant 38 and a GT microsatellite linked to NEDD4L.

52. The kit of claim 50, wherein the position variant is variant 13 at position 82,773 of chromosome 18 of hg8 from the August 6,2001 freeze with coordinates indexed to base 65,000,000.

53. The kit of claim 50, wherein the at least one probe comprises at least on primer, wherein the at least one primer provides direct sequence information about variant 13.

54. The kit of claim 50, wherein the at least one antibody or antibody fragment recognizes an epitope derived from a Ca²⁺-dependent lipid binding C2 domain of NEDD4L.
